(19) United States
(12) Patent Application Publication
(10) Pub. No.: US 2026/0193329 A1
(43) Pub. Date: Jul. 9, 2026

(54) FORMULATIONS FOR ANTI-C1Q ANTIBODIES

(71) Applicant: Annexon, Inc., Brisbane, CA (US)

(72) Inventors: Cecilia Oliyai, Brisbane, CA (US); Khoi Thai, Brisbane, CA (US)

(21) Appl. No.: 19/118,600

(22) PCT Filed: Oct. 6, 2023
(86) PCT No.: PCT/US2023/076250

§ 371 Date: Apr. 4, 2025

Related U.S. Application Data
(60) Provisional application No. 63/414,206, filed on Oct. 7, 2022.

(51) Int. Cl. C07K 16/18; A61K 39/395; A61K 47/12; A61K 47/26

(52) U.S. Cl. CPC C07K 16/18; A61K 39/39591; A61K 47/12; A61K 47/26; C07K 2317/55

(57) ABSTRACT

The present disclosure is generally directed to pharmaceutical compositions comprising anti-C1q antibodies and methods of making and using such compositions. In certain embodiments, such compositions are high concentration anti-C1q antibody formulations that exhibit low viscosity and high stability and can therefore be conveniently delivered to subjects in need thereof with reduced discomfort.

### RELATED APPLICATIONS

This patent application claims priority to U.S. Provisional Patent Application No. 63/414,206, filed Oct. 7, 2022, which is hereby incorporated by reference in its entirety.

### BACKGROUND

Therapeutic macromolecules (e.g., antibodies, antibody fragments, and antibody derivatives) must be formulated in a manner that not only makes the molecules suitable for administration to patients, but also maintains their stability during storage and subsequent use. For example, therapeutic antibodies in liquid solution are prone to degradation, aggregation, or undesired chemical modifications unless the solution is formulated properly. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the viscosity of the solution, the concentration of antibody that can be accommodated by a given formulation, and the visual quality or appearance of the formulation. Thus, when formulating a therapeutic antibody, great care must be taken to arrive at a formulation that remains stable, contains an adequate concentration of antibody, and possesses a suitable viscosity as well as other properties that enable the formulation to be conveniently administered to patients.

Anti-C1q antibodies are one example of a therapeutically relevant macromolecule that requires proper formulation. Anti-C1q antibodies are clinically useful for the treatment or prevention of diseases associated with complement activation such as autoimmune, inflammatory, and neurodegenerative diseases, as well as cancer.

Although anti-C1q antibodies are known, there remains a need in the art for novel pharmaceutical formulations comprising anti-C1q antibodies that are sufficiently stable and suitable for administration to patients.

### SUMMARY

In certain aspects, provided herein are pharmaceutical compositions comprising anti-C1q antibodies, e.g., full-length antibodies, antibody Fab fragments, and/or antibody derivatives, as well as methods of using and making such pharmaceutical compositions. In certain embodiments, the pharmaceutical compositions provided herein comprise a high-concentration anti-C1q antibody solution (whether full-length antibodies, antibody Fab fragments, and/or antibody derivatives) while exhibiting a high agent stability and low viscosity. Such compositions are therefore amenable to delivery, e.g., by intravitreal, subcutaneous, or intravenous injection, to patients suffering from diseases associated with complement pathway. Moreover, the high antibody concentration coupled with low viscosity of compositions provided herein allow for delivery to patients of lower volumes of composition through smaller needles, reducing patient discomfort.

Provided herein is a pharmaceutical composition comprising at least 75 mg/ml of an anti-C1q antibody. The pharmaceutical composition may further comprise from 5 to 25 mM sodium citrate or sodium succinate; from 3% to 7% trehalose; and/or from 0.04% to 0.06% polysorbate 20 or polysorbate 80. The pharmaceutical composition may be at a pH of 6.4-7.5.

In some embodiments, the antibody comprises a light chain variable domain comprising an HVR-L1 having the amino acid sequence of SEQ ID NO: 5, an HVR-L2 having the amino acid of SEQ ID NO: 6, and an HVR-L3 having the amino acid of SEQ ID NO: 7. In some embodiments, the antibody comprises a heavy chain variable domain comprising an HVR-H1 having the amino acid sequence of SEQ ID NO: 9, an HVR-H2 having the amino acid of SEQ ID NO: 10, and an HVR-H3 having the amino acid of SEQ ID NO: 11. The antibody may be a monoclonal antibody, a polyclonal antibody, a recombinant antibody, a humanized antibody, a chimeric antibody, a multispecific antibody, or an antibody derivative thereof, such as a single arm antibody.

In some embodiments, the antibody comprises a light chain variable domain comprising an amino acid sequence with at least about 95% homology to the amino acid sequence selected from SEQ ID NO: 4 and 35-38 and wherein the light chain variable domain comprises an HVR-L1 having the amino acid sequence of SEQ ID NO: 5, an HVR-L2 having the amino acid of SEQ ID NO: 6, and an HVR-L3 having the amino acid of SEQ ID NO: 7. The light chain variable domain may comprise an amino acid sequence selected from SEQ ID NO: 4 and 35-38. In some embodiments, the antibody comprises a heavy chain variable domain comprising an amino acid sequence with at least about 95% homology to the amino acid sequence selected from SEQ ID NO: 8 and 31-34 and wherein the heavy chain variable domain comprises an HVR-H1 having the amino acid sequence of SEQ ID NO: 9, an HVR-H2 having the amino acid of SEQ ID NO: 10, and an HVR-H3 having the amino acid of SEQ ID NO: 11. The heavy chain variable domain may comprise an amino acid sequence selected from SEQ ID NO: 8 and 31-34.

In some embodiments, the antibody is an antibody fragment, such as a Fab fragment, a Fab′ fragment, a F(ab′)2 fragment, a Fv fragment, a diabody, or a single chain antibody molecule. The antibody Fab fragment may preferably comprise a heavy chain Fab fragment of SEQ ID NO: 39. The antibody Fab fragment may preferably comprise a light chain Fab fragment of SEQ ID NO: 40.

In some embodiments, the pharmaceutical composition comprises at least 100 mg/ml, at least 125 mg/ml, at least 150 mg/ml, at least 175 mg/ml, at least 200 mg/ml, at least 225 mg/ml, or at least 250 mg/ml of the anti-C1q antibody. The pharmaceutical composition may comprise 100 mg/ml to 125 mg/ml, 125 mg/ml to 150 mg/ml, 150 mg/ml to 175 mg/ml, 175 mg/ml to 200 mg/ml, or 200 mg/ml to 250 mg/ml of the anti-C1q antibody. In some embodiments, the pharmaceutical composition comprises from 75 mg/ml to 300 mg/ml, from 100 mg/ml to 300 mg/ml, or from 125 mg/ml to 250 mg/ml of the anti-C1q antibody. In some embodiments, the pharmaceutical composition comprises about 75 mg/ml, 100 mg/ml, 125 mg/ml, about 150 mg/ml, about 175 mg/ml, about 200 mg/ml, 225 mg/ml, or about 250 mg/ml of the anti-C1q antibody Fab fragment.

In some embodiments, the pharmaceutical composition comprises about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM sodium citrate or sodium succinate. The pharmaceutical composition may comprise 5 mM to 10 mM, 10 mM to 15 mM, 15 mM to 20 mM, or 20 mM to 25 mM sodium citrate or sodium succinate.

In some embodiments, the pharmaceutical composition is hypertonic or isotonic. In some embodiments, the pharmaceutical composition comprises about 3%, 4%, 5%, 6%, or 7% trehalose. The pharmaceutical composition may comprise 3%-4%, 4%-5%, 5%-6%, or 6%-7% trehalose. In some embodiments, the pharmaceutical composition comprises about 0.04%, 0.05%, or 0.06% polysorbate 20 or polysorbate 80. The pharmaceutical composition may comprise 0.04% to 0.05% or 0.05% to 0.06% polysorbate 20 or polysorbate 80. In some embodiments, the pharmaceutical composition is at a pH of about 6.4, about 6.5, about 6.6, about 6.7, about 6.8, about 6.9, about 7.0, about 7.1, about 7.2, about 7.3, about 7.4, or about 7.5. The pharmaceutical composition may be at a pH in the range of 6.0-6.5 or 6.5-7.0 or 7.0 to 7.5. In some embodiments, the pharmaceutical composition further comprises from 30 mM to 50 mM NaCl. The pharmaceutical composition may comprise about 30 mM, about 35 mM, about 40 mM, about 45 mM, or about 50 mM NaCl. The pharmaceutical composition may comprise 30 mM to 35 mM, 35 mM to 40 mM, 40 mM to 45 mM, or 45 mM to 50 mM NaCl.

In some embodiments, the viscosity of the pharmaceutical composition is no more than 10 cP at 25° C., such as about 1 cP, about 2 cP, about 3 cP, about 4 cP, about 5 cP, about 6 cP, about 7 cP, about 8 cP, about 9 cP, or about 10 cP at 25° C. The viscosity of the pharmaceutical composition may be 1 cp to 5 cp or 5 cp to 10 cp at 25° C.

In some embodiments, the pharmaceutical composition is stable for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks at least 17 weeks, at least 18 weeks, at least 19 weeks, at least 20 weeks, at least 21 weeks, at least 22 weeks, at least 23 weeks, or at least 24 weeks. The pharmaceutical composition may be stable for at least 1 year, at least 1.5 years, or at least two years. The pharmaceutical composition may be stable at a temperature of from −20° C. to 25° C., such as about −20° C., about −15° C., about −10° C., about −5° C., about 0° C., about 4° C., about 5° C., about 10° C., about 15° C., about 20° C., or about 25° C. In some embodiments, the pharmaceutical composition is stable at a temperature of greater than or equal to 25° C., such as between about 25° C. to about 40° C. (e.g., about 25° C., about 30° C., about 35° C., or about 40° C.).

In some embodiments, the pharmaceutical composition is stable after at least one, at least two, at least three, at least four, or at least five freeze/thaw cycles. In some embodiments, the pharmaceutical composition is stable after at least one, at least two, or at least three days of continuous agitation.

In some embodiments, the pharmaceutical composition is suitable for intravitreal or intraocular injection. The pharmaceutical composition may comprise from 5 mM to 20 mM sodium citrate or sodium succinate, such as about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM sodium citrate or sodium succinate.

In some embodiments, the pharmaceutical composition is contained in a syringe. In some embodiments, the pharmaceutical composition is prefilled in a syringe. The syringe may have a fill volume of no more than 300 microliters. In some embodiments, the syringe is configured to deliver a volume of 25-100 microliters. In some embodiments, the syringe is silicon-free and/or low in particulate level. In some embodiments, the pharmaceutical composition is silicon-free and/or low in particulate level. In some embodiments, the prefilled syringe has less than fifty particles per ml of 10 μm particles and/or less than five particles per ml of 25 μm particles. In some embodiments, the pharmaceutical composition has less than fifty particles per ml of 10 μm particles and/or less than five particles per ml of 25 μm particles. In some embodiments, the prefilled syringe is compliant with USP<789> limits. In some embodiments, the pharmaceutical composition is compliant with USP<789> limits.

In some embodiments, the pharmaceutical composition is prepared for subcutaneous injection. The pharmaceutical composition may contained in an injector, autoinjector, or pen. The injector, autoinjector, or pen may have a fill volume of no more than 10 ml.

In some embodiments, the pharmaceutical composition is prepared for intravenous injection. The pharmaceutical composition may be contained in an intravenous solution bag.

In one aspect, disclosed herein is an injector comprising the pharmaceutical composition disclosed herein. In some embodiments, the injector comprises a delivery volume of no more than 10 ml. In some embodiments, the injector is an automatic reusable fixed dose pen. Alternatively, the injector may be an automatic reusable variable dose pen. In some embodiments, the injector is an automatic disposable fixed dose autoinjector. The injector may comprise a delivery volume of no more than 300 microliters. The injector may be a syringe.

In another aspect, described herein is a prefilled syringe comprising a pharmaceutical composition described herein. In some embodiments, the prefilled syringe comprises a delivery volume of no more than 300 microliters. In some embodiments, the prefilled syringe is silicon-free and/or low in particulate level. In some embodiments, the pharmaceutical composition is silicon-free and/or low in particulate level. In some embodiments, the prefilled syringe has less than fifty particles per ml of 10 μm particles and/or less than five particles per ml of 25 μm particles. In some embodiments, the prefilled syringe is compliant with USP<789> limits. In some embodiments, the pharmaceutical composition is compliant with USP<789> limits.

In yet another aspect, described herein is a method of inhibiting synapse loss. The method may comprise administering to a patient suffering from adverse synapse loss the pharmaceutical composition described herein. In some embodiments, the patient has suffered synapse loss as a result of a neurodegenerative disorder, central nervous system disorder, or a peripheral nervous system disorder. The neurodegenerative disorder may be Guillain Barre Syndrome (GBS), amyotrophic lateral sclerosis (ALS) or Huntington's Disease (HD).

In one aspect, disclosed herein is a method of treating or preventing a disease associated with complement activation in an individual in need of such treatment. The method may comprise administering the pharmaceutical composition described herein. The disease associated with complement activation may be a neurodegenerative disorder, which may be associated with loss of synapses or loss nerve connections, associated with synapse loss that is dependent on the complement receptor 3(CR3)/C3 or complement receptor CR1, associated with pathological activity-dependent synaptic pruning, or associated with synapse phagocytosis by microglia. In some embodiments, the neurodegenerative disorder is Alzheimer's disease, amyotrophic lateral sclerosis (ALS), multiple sclerosis, an ophthalmic disorder, glaucoma, myotonic dystrophy, Guillain-Barre' syndrome (GBS), Myasthenia Gravis, Bullous Pemphigoid, spinal muscular atrophy, Down syndrome, Parkinson's disease, traumatic brain injury (TBI), epilepsy, or Huntington's disease (HD). In some embodiments, the neurodegenerative disorder is amyotrophic lateral sclerosis (ALS). In some embodiments, the neurodegenerative disorder is Huntington's disease.

In some embodiments, the disease associated with complement activation is an inflammatory disease, autoimmune disease, complement-associated eye disease or metabolic disorder. The inflammatory disease, autoimmune disease, complement-associated eye disease or metabolic disorder may be selected from diabetes, obesity, rheumatoid arthritis (RA), acute respiratory distress syndrome (ARDS), remote tissue injury after ischemia and reperfusion, complement activation during cardiopulmonary bypass surgery, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobinemia, antiphospholipid syndrome, glaucoma, Chronic open-angle glaucoma, acute closed angle glaucoma, macular degenerative diseases, age-related macular degeneration (AMD), geographic atrophy, choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, ANCA vasculitis, Purtscher retinopathy, Sjogren's dry eye disease, dry AMD, sarcoidosis, temporal arteritis, polyarteritis nodosa, allo-transplantation, hyperacute rejection, hemodialysis, chronic occlusive pulmonary distress syndrome (COPD), asthma, aspiration pneumonia, multiple sclerosis, Multifocal Motor Neuropathy (MMN), Guillain-Barre syndrome, Myasthenia Gravis, Bullous Pemphigoid, or myositis.

The disease associated with complement activation may be an autoimmune disease selected from Multifocal Motor Neuropathy (MMN), Diabetes mellitus type 1, Hashimoto's thyroiditis, Addison's disease, Coeliac disease, Crohn's disease, pernicious anaemia, Pemphigus vulgaris, vitiligo, autoimmune hemolytic anemias, paraneoplastic syndromes, a vasculitis disease, hypocomplementemic urticarial vasculitis (HUV), polymyalgia rheumatica, temporal arteritis, Wegener's granulomatosis, multiple sclerosis, Guillain-Barre syndrome, Myasthenia Gravis, Bullous Pemphigoid, or myositis.

The disease associated with complement activation may be a blood disorder selected from cold agglutinin hemolytic anemia (cold agglutinin disease), cold antibody hemolytic anemia, ABO incompatible acute hemolytic reactions, warm agglutinin hemolytic anemia, warm antibody hemolytic anemia, warm autoimmune hemolytic anemia (WAIHA), autoimmune hemolytic anemia (AIHA) autoimmune thrombocytopenia, antiphospholipid syndrome, Evan's syndrome, neonatal alloimmune thrombocytopenia, red blood cell alloimmunization, Felty's syndrome, antibody-mediated thrombocytopenia, heparin-induced thrombocytopenia (HIT), heparin-induced thrombocytopenia and thrombosis (HITT), thrombotic thrombocytopenic purpura (TTP), immune thrombocytopenic purpura (ITP), thrombocytopenia, thrombosis, vasculitis, lupus nephritis, systemic lupus erythematosus (SLE), glomerulonephritis, anti-phospholipid antibody syndrome (APS), an infection, or a drug-induced hematologic disorder.

### DETAILED DESCRIPTION

### General

Provided herein are a novel pharmaceutical compositions comprising an antibody, antibody fragment, or antibody derivatives, in particular an antibody Fab fragment, capable of binding to C1q, as well as methods of making and using such pharmaceutical compositions. Also provided herein is the use of said pharmaceutical composition to prevent, reduce risk of developing, or treat a disease associated with complement activation.

### Definitions

As used herein the specification, “a” or “an” may mean one or more. As used herein in the claim(s), when used in conjunction with the word “comprising”, the words “a” or “an” may mean one or more than one. For example, reference to an “antibody” is a reference from one to many antibodies. As used herein “another” may mean at least a second or more.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) formed from at least two intact antibodies, antibody fragments so long as they exhibit biological activity, and antibody derivatives. The basic 4-chain antibody unit is a heterotetrameric glycoprotein composed of two identical light (L) chains and two identical heavy (H) chains. The pairing of a V_{H} and V_{L} together forms a single antigen-binding site. For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, CT, 1994, page 71 and Chapter 6. The L chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“α”), delta (“δ”), epsilon (“ε”), gamma (“γ”) and mu (“μ”), respectively. The γ and a classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites. The variable domain mediates antigen binding and defines the specificity of a particular antibody for its particular antigen. However, the variability is not evenly distributed across the entire span of the variable domains. Instead, it is concentrated in three segments called hypervariable regions (HVRs) both in the light-chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

As used herein, the term “CDR” or “complementarity determining region” is intended to mean the non-contiguous antigen binding sites found within the variable region of both heavy and light chain polypeptides. CDRs have been described by Kabat et al., J. Biol. Chem. 252:6609-6616 (1977); Kabat et al., U.S. Dept. of Health and Human Services, “Sequences of proteins of immunological interest” (1991) (also referred to herein as Kabat 1991); by Chothia et al., J. Mol. Biol. 196:901-917 (1987) (also referred to herein as Chothia 1987); and MacCallum et al., J. Mol. Biol. 262:732-745 (1996), where the definitions include overlapping or subsets of amino acid residues when compared against each other. Nevertheless, application of either definition to refer to a CDR of an antibody or grafted antibodies or variants thereof is intended to be within the scope of the term as defined and used herein. As used herein, the terms “CDR-L1”, “CDR-L2”, and “CDR-L3” refer, respectively, to the first, second, and third CDRs in a light chain variable region. As used herein, the terms “CDR-H1”, “CDR-H2”, and “CDR-H3” refer, respectively, to the first, second, and third CDRs in a heavy chain variable region. As used herein, the terms “CDR-1”, “CDR-2”, and “CDR-3” refer, respectively, to the first, second and third CDRs of either chain's variable region.

A number of HVR and CDR delineations are in use and are encompassed herein. The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat numbering) |
| H1 | H31-H35 | H26-H35 | H26-H32 | H30-H35 (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG1 EU antibody. Unless stated otherwise herein, references to residue numbers in the variable domain of antibodies means residue numbering by the Kabat numbering system. Unless stated otherwise herein, references to residue numbers in the constant domain of antibodies means residue numbering by the EU numbering system (e.g., see United States Patent Publication No. 2010-280227).

As used in the present specification, the expression “C1q antibody” should be interpreted as similar to “anti-C1q antibody” and means an antibody capable of binding to C1q. In an embodiment of the present application, the epitope of the antibody is localized into the extracellular domain of the human C1q.

The term “monoclonal antibody” as used herein refers to an antibody obtained from a population of substantially homogeneous antibodies, i.e., the individual antibodies of the population are identical except for possible naturally occurring mutations and/or post-translation modifications (e.g., isomerizations, amidations) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

An “antibody fragment” or “antigen-binding fragment” or “functional fragments” of antibodies comprises a portion of an intact antibody, preferably the antigen binding and/or the variable region of the intact antibody or the F region of an antibody which retains or has modified FcR binding capability. Examples of antibody fragments include Fab, Fab′, F(ab′)₂ and Fv fragments; diabodies; and linear antibodies (see U.S. Pat. No. 5,641,870, Example 2; Zapata et al., Protein Eng. 8(10):1057-1062 (1995)). Additional examples of antibody fragments include antibody derivatives such as single-chain antibody molecules, monovalent antibodies and multispecific antibodies formed from antibody fragments

An “antibody derivative” is any construct that comprises the antigen binding region of an antibody. Examples of antibody derivatives include single-chain antibody molecules, single arm antibodies, antibodies with a single antigen-binding arm, monovalent antibodies and multispecific antibodies formed from antibody fragments.

The term “single-arm antibody” herein is used to cover an antibody that comprises a single antigen-binding arm. The single-arm antibody may comprise an antigen-binding arm and an Fc region, wherein the single antigen-binding arm comprises a light chain variable domain and a heavy chain variable domain; and the Fc region comprises a complex of a first and a second Fc polypeptide. In some embodiments, one but not both of the Fc polypeptides is an N-terminally truncated heavy chain. In some embodiments, the N-terminally truncated heavy chain lacks a heavy chain variable domain, preferably the N-terminally truncated heavy chain lacks a heavy chain constant domain 1. In some embodiments, the antibody may be a bivalent antibody—where one arm binds C1q and the other binds a different antigen. Depending on the other antigen, such an antibody would not crosslink and activate C1q. The single arm antibody may be an antibody with a single antigen-binding arm. “An antibody with a single antigen-binding arm” as used herein means an antibody comprising a single antigen-binding arm and an Fc region, wherein the antigen-binding arm comprises a light chain variable domain and a heavy chain variable domain. In some embodiments, the antibody further comprises an inactive antigen-binding arm, which is incapable of binding to the antigen, or comprises an arm that binds to a different antigen. In some embodiments, the Fc region comprises a complex of a first and a second Fc polypeptide.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments with hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g., from about one to about ten amino acid substitutions, and preferably from about one to about five amino acid substitutions in a native sequence Fc region or in the Fe region of the parent polypeptide. The variant Fc region herein will preferably possess at least about 80% homology with a native sequence Fe region and/or with an Fc region of a parent polypeptide, and most preferably at least about 90% homology therewith, more preferably at least about 95% homology therewith.

As used herein, a “chimeric antibody” refers to an antibody (immunoglobulin) in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Nat'l Acad. Sci. USA, 81:6851-55 (1984)). Chimeric antibodies of interest herein include PRIMATIZED® antibodies wherein the antigen-binding region of the antibody is derived from an antibody produced by, e.g., immunizing macaque monkeys with an antigen of interest. As used herein, “humanized antibody” is a subset of “chimeric antibodies.”

“Humanized” forms of non-human (e.g., murine) antibodies are chimeric antibodies that contain minimal sequence derived from non-human immunoglobulin. In some embodiments, a humanized antibody is a human immunoglobulin (recipient antibody) in which residues from an HVR of the recipient are replaced by residues from an HVR of a non-human species (donor antibody) such as mouse, rat, rabbit or non-human primate having the desired specificity, affinity, and/or capacity. In some instances, FR residues of the human immunoglobulin are replaced by corresponding non-human residues. Furthermore, humanized antibodies may comprise residues that are not found in the recipient antibody or in the donor antibody. These modifications may be made to further refine antibody performance, such as binding affinity. In general, a humanized antibody will comprise substantially all of at least one, and typically two, variable domains, in which all or substantially all of the hypervariable loops correspond to those of a non-human immunoglobulin sequence, and all or substantially all of the FR regions are those of a human immunoglobulin sequence, although the FR regions may include one or more individual FR residue substitutions that improve antibody performance, such as binding affinity, isomerization, immunogenicity, and the like. The number of these amino acid substitutions in the FR is typically no more than 6 in the H chain, and in the L chain, no more than 3. The humanized antibody optionally will also comprise at least a portion of an immunoglobulin constant region (Fc), typically that of a human immunoglobulin. For further details, see, e.g., Jones et al., Nature 321:522-525 (1986); Riechmann et al., Nature 332:323-329 (1988); and Presta, Curr. Op. Struct. Biol. 2:593-596 (1992). See also, for example, Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

A “human antibody” is one that possesses an amino-acid sequence corresponding to that of an antibody produced by a human and/or has been made using any of the techniques for making human antibodies as disclosed herein. This definition of a human antibody specifically excludes a humanized antibody comprising non-human antigen-binding residues. Human antibodies can be produced using various techniques known in the art, including phage-display libraries. Hoogenboom and Winter, J. Mol. Biol., 227:381 (1991); Marks et al., J. Mol. Biol., 222:581 (1991). Also available for the preparation of human monoclonal antibodies are methods described in Cole et al., Monoclonal Antibodies and Cancer Therapy, Alan R. Liss, p. 77 (1985); Boerner et al., J. Immunol., 147(1):86-95 (1991). See also van Dijk and van de Winkel, Curr. Opin. Pharmacol. 5:368-74 (2001). Human antibodies can be prepared by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology). See also, for example, Li et al., Proc. Nat'l Acad Sci. USA, 103:3557-3562 (2006) regarding human antibodies generated via a human B-cell hybridoma technology.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may contain pre-existing amino acid sequence changes. In some embodiments, the number of pre-existing amino acid changes are 10 or fewer, 9 or fewer, 8 or fewer, 7 or fewer, 6 or fewer, 5 or fewer, 4 or fewer, 3 or fewer, or 2 or fewer. Where pre-existing amino acid changes are present in a VH, preferable those changes occur at only three, two, or one of positions 71H, 73H and 78H; for instance, the amino acid residues at those positions may by 71A, 73T and/or 78A. In some embodiments, the V_{L} acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or VH framework sequences. Generally, the selection of human immunoglobulin V_{L} or VH sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include for the VL, the subgroup may be subgroup kappa I, kappa II, kappa III or kappa IV as in Kabat et al., supra. Additionally, for the VH, the subgroup may be subgroup I, subgroup II, or subgroup III as in Kabat et al., supra.

An “amino-acid modification” at a specified position refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

“Identity”, as used herein, indicates that at any particular position in the aligned sequences, the amino acid residue is identical between the sequences. “Similarity”, as used herein, indicates that, at any particular position in the aligned sequences, the amino acid residue is of a similar type between the sequences. For example, leucine may be substituted for isoleucine or valine. Other amino acids which can often be substituted for one another include but are not limited to:
phenylalanine, tyrosine and tryptophan (amino acids having aromatic side chains);
lysine, arginine and histidine (amino acids having basic side chains);
aspartate and glutamate (amino acids having acidic side chains);
asparagine and glutamine (amino acids having amide side chains); and
cysteine and methionine (amino acids having sulphur-containing side chains).

Degrees of identity and similarity can be readily calculated. (See e.g., Computational Molecular Biology, Lesk, A. M., ed., Oxford University Press, New York, 1988; Biocomputing. Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991)

As used herein, an “interaction” between C1q and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

The term “preventing” is art-recognized, and when used in relation to a condition is well understood in the art, and includes administration of a composition which reduces the frequency of, or delays the onset of, symptoms of a medical condition in a subject relative to a subject which does not receive the composition. Thus, prevention of a disease associated with complement activation includes, for example, reducing the incidence of the disease associated with complement activation in a population of patients receiving a prophylactic treatment relative to an untreated control population, and/or delaying the onset of the disease associated with complement activation in a treated population versus an untreated control population, e.g., by a statistically and/or clinically significant amount.

As used herein, the term “specifically recognizes” or “specifically binds” refers to measurable and reproducible interactions such as attraction or binding between a target and an antibody that is determinative of the presence of the target in the presence of a heterogeneous population of molecules including biological molecules. For example, an antibody that specifically or preferentially binds to a target or an epitope is an antibody that binds this target or epitope with greater affinity, avidity, more readily, and/or with greater duration than it binds to other targets or other epitopes of the target. It is also understood that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻, in other instances about 10⁶ M⁻¹ or 10⁷ M⁻, about 10⁸ M⁻¹ to 10⁹ M⁻¹, or about 10¹⁰ M⁻¹ to 10¹¹ M or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

The term “subject” as used herein refers to a living mammal and may be interchangeably used with the term “patient”. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

The term “therapeutically effective amount” of a compound with respect to the subject method of treatment refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment. A therapeutically effective amount herein may vary according to factors such as the disease state, age, sex, and weight of the patient, and the ability of the antibody to elicit a desired response in the individual.

As used herein, the term “treating” or “treatment” includes reducing, arresting, or reversing the symptoms, clinical signs, or underlying pathology of a condition to stabilize or improve a subject's condition or to reduce the likelihood that the subject's condition will worsen as much as if the subject did not receive the treatment.

### Antibodies

All sequences mentioned in the present disclosure are incorporated by reference from U.S. patent application Ser. No. 14/933,517, U.S. patent application Ser. No. 14/890,811, U.S. Pat. Nos. 8,877,197, 9,708,394, U.S. patent application Ser. No. 15/360,549, U.S. Pat. Nos. 9,562,106, 10,450,382, 10,457,745, International Patent Application No. PCT/US2018/022462 each of which is hereby incorporated by reference for the antibodies and related compositions that it discloses.

Antibodies may be prepared by the use of recombinant DNA engineering techniques. Such engineered versions include those created, for example, from natural antibody variable regions by insertions, deletions or changes in or to the amino acid sequences of the natural antibodies. Particular examples of this type include those engineered variable region domains containing at least one CDR and optionally one or more framework amino acids from one antibody and the remainder of the variable region domain from a second antibody. The DNA encoding the antibody may be prepared by deleting all but the desired portion of the DNA that encodes the antibody. DNA encoding chimerized antibodies may be prepared by recombining DNA substantially or exclusively encoding human constant regions and DNA encoding variable regions derived substantially or exclusively from the sequence of the variable region of a mammal other than a human. DNA encoding humanized antibodies may be prepared by recombining DNA encoding constant regions and variable regions other than the complementarity determining regions (CDRs) derived substantially or exclusively from the corresponding human antibody regions and DNA encoding CDRs derived substantially or exclusively from a mammal other than a human.

Suitable sources of DNA molecules that encode antibodies include cells, such as hybridomas, that express the full length antibody. For example, the antibody may be isolated from a host cell that expresses an expression vector that encodes the heavy and/or light chain of the antibody.

Antibody fragments, including but not limited to Fab fragments, and/or antibody derivatives may also be prepared by the use of recombinant DNA engineering techniques involving the manipulation and re-expression of DNA encoding antibody variable and constant regions. Standard molecular biology techniques may be used to modify, add or delete further amino acids or domains as desired. Any alterations to the variable or constant regions are still encompassed by the terms ‘variable’ and ‘constant’ regions as used herein. In some instances, PCR is used to generate an antibody fragment by introducing a stop codon immediately following the codon encoding the interchain cysteine of C_{H}1, such that translation of the C_{H}1 domain stops at the interchain cysteine. Methods for designing suitable PCR primers are well known in the art and the sequences of antibody C_{H}1 domains are readily available. In some embodiments, stop codons may be introduced using site-directed mutagenesis techniques.

An antibody of the present disclosure may be derived from any antibody isotype (“class”) including for example IgG, IgM, IgA, IgD and IgE and subclasses thereof, including for example IgG1, IgG2, IgG3 and IgG4. In certain preferred embodiments, the heavy and light chains of the antibody are from IgG. The heavy and/or light chains of the antibody may be from murine IgG or human IgG. In certain other preferred embodiments, the heavy and/or light chains of the antibody are from human IgG1. In still other preferred embodiments, the heavy and/or light chains of the antibody are from human IgG4.

An antibody of the present disclosure may bind to and inhibit a biological activity of C1q, C1r, or C1s. For example, (1) C1q binding to an autoantibody, (2) C1q binding to C1r, (3) C1q binding to C1s, (4) C1q binding to phosphatidylserine, (5) C1q binding to pentraxin-3, (6) C1q binding to C-reactive protein (CRP), (7) C1q binding to globular C1q receptor (gC1qR), (8) C1q binding to complement receptor 1 (CR1), (9) C1q binding to B-amyloid, or (10) C1q binding to calreticulin. In other embodiments, the biological activity of C1q is (1) activation of the classical complement activation pathway, (2) reduction in lysis and/or reduction in C3 deposition, (3) activation of antibody and complement dependent cytotoxicity, (4) CH50 hemolysis, (5) a reduction in red blood cell lysis, (6) a reduction in red blood cell phagocytosis, (7) a reduction in dendritic cell infiltration, (8) inhibition of complement-mediated red blood cell lysis, (9) a reduction in lymphocyte infiltration, (10) a reduction in macrophage infiltration, (11) a reduction in antibody deposition, (12) a reduction in neutrophil infiltration, (13) a reduction in platelet phagocytosis, (14) a reduction in platelet lysis, (15) an improvement in transplant graft survival, (16) a reduction in macrophage mediated phagocytosis, (17) a reduction in autoantibody mediated complement activation, (18) a reduction in red blood cell destruction due to transfusion reactions, (19) a reduction in red blood cell lysis due to alloantibodies, (20) a reduction in hemolysis due to transfusion reactions, (21) a reduction in alloantibody mediated platelet lysis, (22) an improvement in anemia, (23) a reduction in eosinophilia, (24) a reduction in C3 deposition on red blood cells (e.g., a reduction of deposition of C3b, iC3b, etc., on RBCs), (25) a reduction in C3 deposition on platelets (e.g., a reduction of deposition of C3b, iC3b, etc., on platelets), (26) reduction in anaphylatoxin production, (27) a reduction in autoantibody mediated blister formation, (28) a reduction in autoantibody induced erythematosus, (29) a reduction in red blood cell destruction due to transfusion reactions, (30) a reduction in platelet lysis due to transfusion reactions, (31) a reduction in mast cell activation, (32) a reduction in mast cell histamine release, (33) a reduction in vascular permeability, (34) a reduction in complement deposition on transplant graft endothelium, (35) B-cell antibody production, (36) dendritic cell maturation, (37) T-cell proliferation, (38) cytokine production, (39) microglia activation, (40) Arthus reaction, (41) a reduction of anaphylatoxin generation in transplant graft endothelium, or (42) activation of complement receptor 3 (CR3/C3) expressing cells.

In some embodiments, CH50 hemolysis comprises human, mouse, and/or rat CH50 hemolysis. In some embodiments, the antibody is capable of neutralizing from at least about 50%, to at least about 95% of CH50 hemolysis. In some embodiments, the antibody is capable of neutralizing 50%, 60%, 70%, 80, 90%, or 100% of CH50 hemolysis. The antibody may also be capable of neutralizing at least 50% of CH50 hemolysis at a dose of less than 150 ng/ml, less than 100 ng/ml, less than 50 ng/ml, or less than 20 ng/ml.

Other in vitro assays to measure complement activity include ELISA assays for the measurement of split products of complement components or complexes that form during complement activation. Complement activation via the classical pathway can be measured by following the levels of C4d and C4 in the serum. Activation of the alternative pathway can be measured in an ELISA by assessing the levels of Bb or C3bBbP complexes in circulation. An in vitro antibody-mediated complement activation assay may also be used to evaluate inhibition of C3a production.

An antibody of the present disclosure may be a monoclonal antibody, a polyclonal antibody, a recombinant antibody, a humanized antibody, a human antibody, a chimeric antibody, a multispecific antibody, an antibody fragment thereof, or a derivative thereof, including a single arm antibody. In some embodiments, the antibody is humanized antibody.

The antibodies of the present disclosure may also be an antibody fragment, such as a Fab fragment, a Fab′ fragment, a F(ab′)₂ fragment, a Fv fragment, a diabody, or a single chain antibody molecule. In some embodiments, the antibody fragment is a Fab fragment.

In some embodiments, antibodies are human monoclonal antibodies which may be prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable and constant regions derived from human germline and/or non-germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

In some embodiments, antibodies are humanized and/or chimeric monoclonal antibodies, which can be raised by immunizing rodents (e.g., mice, rats, hamsters and guinea pigs) with either (1) the native complement component (e.g., C1q) derived from enzymatic digestion of a purified complement component from human plasma or serum, or (2) a recombinant complement component, or its derived fragment, expressed by either eukaryotic or prokaryotic systems. Other animals can be used for immunization, e.g., non-human primates, transgenic mice expressing human immunoglobulins, and severe combined immunodeficient (SCID) mice transplanted with human B-lymphocytes.

Polyclonal and monoclonal antibodies are naturally generated as immunoglobulin (Ig) molecules in the immune system's response to a pathogen. A dominating format with a concentration of 8 mg/ml in human serum, the ~150-kDa IgG1 molecule is composed of two identical ~50-kDa heavy chains and two identical ~25-kDa light chains.

Hybridomas can be generated by conventional procedures by fusing B-lymphocytes from the immunized animals with myeloma cells. In addition, anti-C1q antibodies can be generated by screening recombinant single-chain Fv or Fab libraries from human B-lymphocytes in a phage-display system. The specificity of the MAbs to human C1q can be tested by enzyme linked immunosorbent assay (ELISA), Western immunoblotting, or other immunochemical techniques.

The inhibitory activity on complement activation of antibodies identified in the screening process can be assessed by hemolytic assays using either unsensitized rabbit or guinea pig RBCs for the alternative complement pathway, or sensitized chicken or sheep RBCs for the classical complement pathway. Those hybridomas that exhibit an inhibitory activity specific for the classical complement pathway are cloned by limiting dilution. The antibodies are purified for characterization for specificity to human C1q by the assays described above.

### Anti-Complement C1q Antibodies

The anti-C1q antibodies disclosed herein are potent inhibitors of C1q and can be dosed for continuous inhibition of C1q function over any period, and then optionally withdrawn to allow for return of normal C1q function at times when its activity may be important.

C1q is a large multimeric protein of 460 kDa consisting of 18 polypeptide chains (6 C1q A chains, 6 C1q B chains, and 6 C1q C chains). C1r and C1s complement proteins bind to the C1q tail region to form the C1 complex (C1qr₂s₂).

The antibodies of this disclosure specifically recognize complement factor C1q and/or C1q in the C1 complex of the classical complement activation pathway. The bound complement factor may be derived, without limitation, from any organism having a complement system, including any mammalian organism such as human, mouse, rat, rabbit, monkey, dog, cat, cow, horse, camel, sheep, goat, or pig.

As used herein “C1 complex” refers to a protein complex that may include, without limitation, one C1q protein, two C1r proteins, and two C1s proteins (e.g., C1qr²s²).

Anti-C1q antibodies disclosed herein may inhibit C1 complex formation.

As used herein “complement factor C1q” refers to both wild type sequences and naturally occurring variant sequences.

A non-limiting example of a complement factor C1q recognized by antibodies of this disclosure is human C1q, including the three polypeptide chains A, B, and C:
C1q, chain A (Homo sapiens), Accession No. Protein Data Base: NP_057075.1; GenBank No.: NM_015991:
>gi|7705753|ref|NP_057075.1 complement C1q subcomponent subunit A precursor [Homo sapiens]

(SEQ ID NO: 1)
MEGPRGWLVLCVLAISLASMVTEDLCRAPDGKKGEAGRPGRRGRPGLKG
EQGEPGAPGIRTGIQGLKGDQGEPGPSGNPGKVGYPGPSGPLGARGIPG
IKGTKGSPGNIKDQPRPAFSAIRRNPPMGGNVVIFDTVITNQEEPYQNH
SGRFVCTVPGYYYFTFQVLSQWEICLSIVSSSRGQVRRSLGFCDTTNKG
LFQVVSGGMVLQLQQGDQVWVEKDPKKGHIYQGSEADSVFSGFLIFPS
A.

C1q, chain B (Homo sapiens), Accession No. Protein Data Base: NP_000482.3; GenBank No.: NM 000491.3:
>gi|87298828|ref|NP_000482.3|complement C1q subcomponent subunit B precursor [Homo sapiens]

(SEQ ID NO: 23)
MMMKIPWGSIPVLMLLLLLGLIDISQAQLSCTGPPAIPGIPGIPGTPGP
DGQPGTPGIKGEKGLPGLAGDHGEFGEKGDPGIPGNPGKVGPKGPMGPK
GGPGAPGAPGPKGESGDYKATQKIAFSATRTINVPLRRDQTIRFDHVIT
NMNNNYEPRSGKFTCKVPGLYYFTYHASSRGNLCVNLMRGRERAQKVVT
FCDYAYNTFQVTTGGMVLKLEQGENVFLQATDKNSLLGMEGANSIFSGF
LLFPDMEA.

C1q, chain C (Homo sapiens), Accession No. Protein Data Base: NP_001107573.1; GenBank No.: NM_001114101.1:
>gi|166235903|ref|NP001107573.1|complement C1q subcomponent subunit C precursor [Homo sapiens]

(SEQ ID NO: 24)
MDVGPSSLPHLGLKLLLLLLLLPLRGQANTGCYGIPGMPGLPGAPGKDG
YDGLPGPKGEPGIPAIPGIRGPKGQKGEPGLPGHPGKNGPMGPPGMPGV
PGPMGIPGEPGEEGRYKQKFQSVFTVTRQTHQPPAPNSLIRFNAVLTNP
QGDYDTSTGKFTCKVPGLYYFVYHASHTANLCVLLYRSGVKVVTFCGHT
SKTNQVNSGGVLLRLQVGEEVWLAVNDYYDMVGIQGSDSVFSGFLLFP
D.

Accordingly, an anti-C1q antibody of the present disclosure may bind to polypeptide chain A, polypeptide chain B, and/or polypeptide chain C of a C1q protein. In some embodiments, an anti-C1q antibody of the present disclosure binds to polypeptide chain A, polypeptide chain B, and/or polypeptide chain C of human C1q or a homolog thereof, such as mouse, rat, rabbit, monkey, dog, cat, cow, horse, camel, sheep, goat, or pig C1q. In some embodiments, the anti-C1q antibody is a human antibody, a humanized antibody, a chimeric antibody, or a fragment thereof or a derivative thereof. In some embodiments, the antibody is humanized antibody. In some embodiments, the antibody is antibody fragment, such as, a Fab fragment.

All sequences mentioned in the following twenty paragraphs are incorporated by reference from U.S. Pat. No. 9,708,394, which is hereby incorporated by reference for the antibodies and related compositions that it discloses.

Light Chain and Heavy Chain Variable Domain Sequences of Antibody M1 (Mab1), Incorporated by Reference from U.S. Pat. No. 9,708,394

Using standard techniques, the nucleic acid and amino acid sequences encoding the light chain variable and the heavy chain variable domain of antibody M1 were determined. The amino acid sequence of the light chain variable domain of antibody M1 is:

(SEQ ID NO: 4)
DVQITQSPSYLAASPGETITINCRASKSINKYLAWYQEKPGKTNKLLIY
SGSTLQSGIPSRFSGSGSGTDFTLTISSLEPEDFAMYYCQQHNEYPLTF
GAGTKLELK.

The hyper variable regions (HVRs) of the light chain variable domain are depicted in bolded and underlined text. In some embodiments, the HVR-L1 of the M1 light chain variable domain has the sequence RASKSINKYLA (SEQ ID NO:5), the HVR-L2 of the M1 light chain variable domain has the sequence SGSTLQS (SEQ ID NO:6), and the HVR-L3 of the M1 light chain variable domain has the sequence QQHNEYPLT (SEQ ID NO:7).

The amino acid sequence of the heavy chain variable domain of antibody M1 is:

(SEQ ID NO: 8)
QVQLQQPGAELVKPGASVKLSCKSSGYHFTSYWMHWVKQRPGQGLEWIG
VIHPNSGSINYNEKFESKATLTVDKSSSTAYMQLSSLTSEDSAVYYCAG
ERDSTEVLPMDYWGQGTSVTVSS.

The hyper variable regions (HVRs) of the heavy chain variable domain are depicted in bolded and underlined text. In some embodiments, the HVR-H1 of the M1 heavy chain variable domain has the sequence GYHFTSYWMH (SEQ ID NO:9), the HVR-H2 of the M1 heavy chain variable domain has the sequence VIHPNSGSINYNEKFES (SEQ ID NO:10), and the HVR-H3 of the M1 heavy chain variable domain has the sequence ERDSTEVLPMDY (SEQ ID NO:11).

The nucleic acid sequence encoding the light chain variable domain was determined to be:

(SEQ ID NO: 12)
GATGTCCAGATAACCCAGTCTCCATCTTATCTTGCTGCATCTCCTGGAG
AAACCATTACTATTAATTGCAGGGCAAGTAAGAGCATTAACAAATATTT
AGCCTGGTATCAAGAGAAACCTGGGAAAACTAATAAGCTTCTTATCTAC
TCTGGATCCACTTTGCAATCTGGAATTCCATCAAGGTTCAGTGGCAGTG
GATCTGGTACAGATTTCACTCTCACCATCAGTAGCCTGGAGCCTGAAGA
TTTTGCAATGTATTACTGTCAACAACATAATGAATACCCGCTCACGTTC
GGTGCTGGGACCAAGCTGGAGCTGAAA.

The nucleic acid sequence encoding the heavy chain variable domain was determined to be:

(SEQ ID NO: 13)
CAGGTCCAACTGCAGCAGCCTGGGGCTGAGCTGGTAAAGCCTGGGGCTT
CAGTGAAGTTGTCCTGCAAGTCTTCTGGCTACCATTTCACCAGCTACTG
GATGCACTGGGTGAAGCAGAGGCCTGGACAAGGCCTTGAGTGGATTGGA
GTGATTCATCCTAATAGTGGTAGTATTAACTACAATGAGAAGTTCGAGA
GCAAGGCCACACTGACTGTAGACAAATCCTCCAGCACAGCCTACATGCA
ACTCAGCAGCCTGACATCTGAGGACTCGGCGGTCTATTATTGTGCAGGA
GAGAGAGATTCTACGGAGGTTCTCCCTATGGACTACTGGGGTCAAGGAA
CCTCAGTCACCGTCTCCTCA.

The Mab1-Fab is the Fab of the Mab1 (M1) antibody.

### DEPOSIT OF MATERIAL

The following materials have been deposited according to the Budapest Treaty in the American Type Culture Collection, ATCC Patent Depository, 10801 University Blvd., Manassas, Va. 20110-2209, USA (ATCC):

| | | Deposit ATCC | |
|---|---|---|---|
| Sample ID | Isotype | Date | Accession No. |
| Mouse hybridoma C1qM1 | IgG1, | Jun. 6, | PTA-120399 |
| 7788-1(M) 051613 producing | kappa | 2013 | |
| anti-C1q antibody M1 | | | |

The hybridoma cell line producing the M1 antibody (mouse hybridoma C1qM1 7788-1(M) 051613) has been deposited with ATCC under conditions that assure that access to the culture will be available during pendency of the patent application and for a period of 30 years, or 5 years after the most recent request, or for the effective life of the patent, whichever is longer. A deposit will be replaced if the deposit becomes nonviable during that period. The deposit is available as required by foreign patent laws in countries wherein counterparts of the subject application, or its progeny are filed. However, it should be understood that the availability of the deposit does not constitute a license to practice the subject invention in derogation of patent rights granted by governmental action.

Humanized Anti-Complement C1q Antibodies, (Incorporated by Reference from U.S. Pat. No. 10,316,081

Humanized antibodies of the present disclosure specifically bind to a complement factor C1q and/or C1q protein in the C1 complex of the classical complement pathway. The humanized anti-C1q antibody may specifically bind to human C1q, human and mouse C1q, to rat C1q, or human C1q, mouse C1q, and rat C1q.

In some embodiments, the human heavy chain constant region is a human IgG4 heavy chain constant region comprising the amino acid sequence of SEQ ID NO:26, or with at least 70%, at least about 75%, at least about 80%, at least about 85%, at least about 90% homology to SEQ ID NO: 26. The human IgG4 heavy chain constant region may comprise an Fc region with one or more modifications and/or amino acid substitutions according to Kabat numbering. In such cases, the Fc region comprises a leucine to glutamate amino acid substitution at position 248, wherein such a substitution inhibits the Fc region from interacting with an Fc receptor. In some embodiments, the Fc region comprises a serine to proline amino acid substitution at position 241, wherein such a substitution prevents arm switching in the antibody.

The amino acid sequence of human IgG4 (S241P L248E; that is corresponding to S108P and L115E in SEQ ID NO: 26) heavy chain constant domain is:

(SEQ ID NO: 26)
ASTKGPSVFPLAPCSRSTSESTAALGCLVKDYFPEPVTVSWNSGALTSG
VHTFPAVLQSSGLYSLSSVVTVPSSSLGTKTYTCNVDHKPSNTKVDKRV
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

The antibody may comprise a heavy chain variable domain and a light chain variable domain, wherein the heavy chain variable domain comprises an amino acid sequence selected from any one of SEQ ID NOs: 31-34, or an amino acid sequence with at least about 90% homology to the amino acid sequence selected from any one of SEQ ID NOs: 31-34. In certain such embodiments, the light chain variable domain comprises an amino acid sequence selected from any one of SEQ ID NOs: 35-38, or an amino acid sequence with at least about 90% homology to the amino acid sequence selected from any one of SEQ ID NOs: 35-38.

The amino acid sequence of heavy chain variable domain variant 1 (VH1) is:

(SEQ ID NO: 31)
QVQLVQSGAELKKPGASVKVSCKSSGYHFTSYWMHWVKQAPGQGLEWIG
VIHPNSGSINYNEKFESKATITVDKSTSTAYMQLSSLTSEDSAVYYCAG
ERDSTEVLPMDYWGQGTSVTVSS.

The hyper variable regions (HVRs) of VH1 are depicted in bolded and underlined text.

The amino acid sequence of heavy chain variable domain variant 2 (VH2) is:

(SEQ ID NO: 32)
QVQLVQSGAELKKPGASVKVSCKSSGYHFTSYWMHWVKQAPGQGLEWIG
VIHPNSGSINYNEKFESRATITVDKSTSTAYMELSSLRSEDTAVYYCAG
ERDSTEVLPMDYWGQGTTVTVSS.

The hyper variable regions (HVRs) of VH2 are depicted in bolded and underlined text.

The amino acid sequence of heavy chain variable domain variant 3 (VH3) is:

(SEQ ID NO: 33)
QVQLVQSGAELKKPGASVKVSCKSSGYHFTSYWMHWVKQAPGQGLEWIG
VIHPNSGSINYNEKFESRVTITVDKSTSTAYMELSSLRSEDTAVYYCAG
ERDSTEVLPMDYWGQGTTVTVSS.

The hyper variable regions (HVRs) of VH3 are depicted in bolded and underlined text.

The amino acid sequence of heavy chain variable domain variant 4 VH4 is:

(SEQ ID NO: 34)
QVQLVQSGAELKKPGASVKVSCKSSGYHFTSYWMHWVRQAPGQGLEWIG
VIHPNSGSINYNEKFESRVTITVDKSTSTAYMELSSLRSEDTAVYYCAG
ERDSTEVLPMDYWGQGTTVTVSS.

The hyper variable regions (HVRs) of VH4 are depicted in bolded and underlined text.

The amino acid sequence of kappa light chain variable domain variant 1 (Vκ1) is:

(SEQ ID NO: 35)
DVQITQSPSYLAASLGERATINCRASKSINKYLAWYQQKPGKTNKLLIY
SGSTLQSGIPARFSGSGSGTDFTLTISSLEPEDFAMYYCQQHNEYPLTF
GQGTKLEIK.

The hyper variable regions (HVRs) of Vκ1 are depicted in bolded and underlined text.

The amino acid sequence of kappa light chain variable domain variant 2 (Vκ2) is:

(SEQ ID NO: 36)
DVQITQSPSSLSASLGERATINCRASKSINKYLAWYQQKPGKANKLLIY
SGSTLQSGIPARFSGSGSGTDFTLTISSLEPEDFAMYYCQQHNEYPLTF
GQGTKLEIK.

The hyper variable regions (HVRs) of Vκ2 are depicted in bolded and underlined text. The amino acid sequence of kappa light chain variable domain variant 3 (Vκ3) is:

(SEQ ID NO: 37)
DVQITQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIK.

The hyper variable regions (HVRs) of Vκ3 are depicted in bolded and underlined text.

The amino acid sequence of kappa light chain variable domain variant 4 (Vκ4) is:

(SEQ ID NO: 38)
DIQLTQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIK.

The hyper variable regions (HVRs) of Vκ4 are depicted in bolded and underlined text.

The antibody may comprise a light chain variable domain amino acid sequence that is at least 85%, 90%, or 95% identical to SEQ ID NO:35-38 while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:31-34 while retaining the HVR-H1 GYHFTSYWMH (SEQ ID NO:9), the HVR-H2 VIHPNSGSINYNEKFES (SEQ ID NO: 10), and the HVR-H3 ERDSTEVLPMDY (SEQ ID NO:11).

In some embodiments, the antibody comprises a light chain variable domain amino acid sequence of SEQ ID NO: 35 and a heavy chain variable domain amino acid sequence of SEQ ID NO: 31. In some embodiments, the antibody comprises a light chain variable domain amino acid sequence of SEQ ID NO: 36 and a heavy chain variable domain amino acid sequence of SEQ ID NO: 32. In some embodiments, the antibody comprises a light chain variable domain amino acid sequence of SEQ ID NO: 37 and a heavy chain variable domain amino acid sequence of SEQ ID NO: 33. In some embodiments, the antibody comprises a light chain variable domain amino acid sequence of SEQ ID NO: 38 and a heavy chain variable domain amino acid sequence of SEQ ID NO: 34.

The full-length antibody Mab2 comprises the heavy chain variable domain variant 3 (VH3)(SEQ ID NO: 33) and the kappa light chain variable domain variant 3 (Vκ3) (SEQ Id NO: 37). The Mab2-Fab is the Fab of the Mab2 antibody.

The antibody may comprise a heavy chain and a light chain, wherein the heavy chain comprises the amino acid sequence of SEQ ID NO: 14; and the light chain comprises the amino acid sequence of SEQ ID NO: 40.

The amino acid sequence of the heavy chain is:

(SEQ ID NO: 14)
QVQLVQSGAELKKPGASVKVSCKSSGYHFTSYWMHWVKQAPGQGLEWIG
VIHPNSGSINYNEKFESRVTITVDKSTSTAYMELSSLRSEDTAVYYCAG
ERDSTEVLPMDYWGQGTTVTVSSASTKGPSVFPLAPCSRSTSESTAALG
CLVKDYFPEPVTVSWNSGALTSGVHTFPAVLQSSGLYSLSSVVTVPSSS
LGTKTYTCNVDHKPSNTKVDKRVESKYGPPCPPCPAPEFEGGPSVFLFP
PKPKDTLMISRTPEVTCVVVDVSQEDPEVQFNWYVDGVEVHNAKTKPRE
EQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNY
KTTPPVLDSDGSFFLYSRLTVDKSRWQEGNVFSCSVMHEALHNHYTQKS
LSLSLGK.

The hyper variable regions (HVRs) of VH3 are depicted in bolded and underlined text.

The amino acid sequence of the light chain is:

(SEQ ID NO: 40)
DVQITQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The complementarity determining regions (CDRs) of SEQ ID NO:40 are depicted in bolded and underlined text.

In some embodiments, humanized anti-C1q antibodies of the present disclosure include a heavy chain variable region that contains an Fab region and a heavy chain constant regions that contains an Fc region, where the Fab region specifically binds to a C1q protein of the present disclosure, but the Fc region is incapable of binding the C1q protein. In some embodiments, the Fc region is from a human IgG1, IgG2, IgG3, or IgG4 isotype. In some embodiments, the Fc region is incapable of inducing complement activity and/or incapable of inducing antibody-dependent cellular cytotoxicity (ADCC). In some embodiments, the Fc region comprises one or more modifications, including, without limitation, amino acid substitutions. In certain embodiments, the Fc region of humanized anti-C1q antibodies of the present disclosure comprise an amino acid substitution at position 248 according to Kabat numbering convention or a position corresponding to position 248 according to Kabat numbering convention, and/or at position 241 according to Kabat numbering convention or a position corresponding to position 241 according to Kabat numbering convention. In some embodiments, the amino acid substitution at position 248 or a positions corresponding to position 248 inhibits the Fc region from interacting with an Fc receptor. In some embodiments, the amino acid substitution at position 248 or a positions corresponding to position 248 is a leucine to glutamate amino acid substitution. In some embodiments, the amino acid substitution at position 241 or a positions corresponding to position 241 prevents arm switching in the antibody. In some embodiments, the amino acid substitution at position 241 or a positions corresponding to position 241 is a serine to proline amino acid substitution. In certain embodiments, the Fc region of humanized anti-C1q antibodies of the present disclosure comprises the amino acid sequence of SEQ ID NO: 47, or an amino acid sequence with at least about 70%, at least about 75%, at least about 80% at least about 85% at least about 90%, or at least about 95% homology to the amino acid sequence of SEQ ID NO: 47.

### Anti-C1q Fab Fragment (e.g., FabA)

All anti-C1q antibody Fab fragment sequences are incorporated by reference from U.S. Pat. No. 10,723,788, which is hereby incorporated by reference for the antibodies and related compositions that it discloses.

In certain embodiments, the present disclosure provides an anti-C1q antibody Fab fragment that binds to a C1q protein comprising a heavy (V_{H}/C_{H}1) and light chain (VL/C_{L}), wherein the anti-C1q antibody Fab fragment has six complementarity determining regions (CDRs), three each from V_{L} and V_{H} (HCDR1, HCDR2, HCDR3, and LCDR1, LCDR2, LCDR3). The heavy chain of the antibody Fab fragment is truncated after the first heavy chain domain of IgG1 (SEQ ID NO: 39), and comprises the following amino acid sequence:

(SEQ ID NO: 39)
QVQLVQSGAELKKPGASVKVSCKSSGYHFTSYWMHWVKQAPGQGLEWIG
VIHPNSGSINYNEKFESRVTITVDKSTSTAYMELSSLRSEDTAVYYCAG
ERDSTEVLPMDYWGQGTTVTVSSASTKGPSVFPLAPSSKSTSGGTAALG
CLVKDYFPEPVTVSWNSGALTSGVHTFPAVLQSSGLYSLSSVVTVPSSS
LGTQTYICNVNHKPSNTKVDKKVEPKSCDKTHT

The complementarity determining regions (CDRs) of SEQ ID NO:39 are depicted in bolded and underlined text.

The light chain domain of the antibody Fab fragment comprises the following amino acid sequence (SEQ ID NO: 40):

(SEQ ID NO: 40)
DVQITQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The complementarity determining regions (CDRs) of SEQ ID NO:40 are depicted in bolded and underlined text.

The FabA is an anti-C1q antibody Fab fragment comprising the heavy chain domain comprising SEQ ID NO: 39 and the light chain domain comprising SEQ ID NO: 40.

The Mab1-Fab is the Fab of the Mab1 (M1) antibody.

The Mab2-Fab is the Fab of the Mab2 antibody.

### Anti-C1q Single-Arm Antibody

All anti-C1q single arm antibody sequences disclosed in U.S. Pat. App. No. 63/288,883 and U.S. Pat. App. No. 63/288,885, which are hereby incorporated by reference for the antibodies and related compositions they disclose.

In certain aspects, the present disclosure provides an antibody that binds to a protein in the complement cascade, such as a C1q protein. The antibody that binds to C1q comprises a single C1q antigen-binding arm and an Fc region. The single C1q antigen-binding arm may comprise a light chain variable domain and a heavy chain variable domain. The Fc region may comprise a complex of a first and a second Fc polypeptide. The Fc region may comprise a Fcγ receptor binding site mutation. The antibody may be of the IgG4 class. In some embodiments, one but not both of the Fc polypeptide is an N-terminally truncated heavy chain. In some embodiments, the Fcγ receptor is FcγRI, FcγRII, or FcγRIII, preferably FcγRI. The Fcγ receptor binding site mutation may comprise a IgG4 L115E mutation.

(SEQ ID NO: 40)
DVQITQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC

The complementarity determining regions (CDRs) of SEQ ID NO: 40 are depicted in bolded and underlined text. In some embodiments, the HVR-L1 of the light chain variable domain has the sequence RASKSINKYLA (SEQ ID NO:5), the HVR-L2 of the light chain variable domain has the sequence SGSTLQS (SEQ ID NO:6), and the HVR-L3 of the light chain variable domain has the sequence QQHNEYPLT (SEQ ID NO:7).

The light chain of the single-arm antibody may comprise the following light chain variable domain amino acid sequence:

(SEQ ID NO: 4)
DVQITQSPSYLAASPGETITINCRASKSINKYLAWYQEKPGKTNKLLIY
SGSTLQSGIPSRFSGSGSGTDFTLTISSLEPEDFAMYYCQQHNEYPLTF
GAGTKLELK.

The single-arm antibody may comprise a light chain variable domain amino acid sequence that is at least 85%, 90%, or 95% identical to SEQ ID NO: 10, preferably while retaining the HVR-L1 RASKSINKYLA (SEQ ID NO: 5), the HVR-L2 SGSTLQS (SEQ ID NO: 6), and the HVR-L3 QQHNEYPLT (SEQ ID NO: 7).

The single-arm antibody may comprise the following amino acid sequence of kappa light chain variable domain variant 1 (Vκ1):

(SEQ ID NO: 35)
DVQITQSPSYLAASLGERATINCRASKSINKYLAWYQQKPGKTNKLLIY
SGSTLQSGIPARFSGSGSGTDFTLTISSLEPEDFAMYYCQQHNEYPLTF
GQGTKLEIK.

The hyper variable regions (HVRs) of Vκ1 are depicted in bolded and underlined text.

The single-arm antibody may comprise the following amino acid sequence of kappa light chain variable domain variant 2 (Vκ2):

(SEQ ID NO: 36)
DVQITQSPSSLSASLGERATINCRASKSINKYLAWYQQKPGKANKLLIY
SGSTLQSGIPARFSGSGSGTDFTLTISSLEPEDFAMYYCQQHNEYPLTF
GQGTKLEIK.

The hyper variable regions (HVRs) of Vκ2 are depicted in bolded and underlined text.

The single-arm antibody may comprise the following amino acid sequence of kappa light chain variable domain variant 3 (Vκ3):

(SEQ ID NO: 37)
DVQITQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIK.

The hyper variable regions (HVRs) of Vκ3 are depicted in bolded and underlined text.

The single-arm antibody may comprise the following amino acid sequence of kappa light chain variable domain variant 4 (Vκ4):

(SEQ ID NO: 38)
DIQLTQSPSSLSASLGERATINCRASKSINKYLAWYQQKPGKAPKLLIY
SGSTLQSGIPARFSGSGSGTDFTLTISSLEPEDFAMYYCQQHNEYPLTF
GQGTKLEIK.

The hyper variable regions (HVRs) of Vκ4 are depicted in bolded and underlined text.

The single-arm antibody may comprise a light chain variable domain amino acid sequence that is at least 85%, 90%, or 95% identical to SEQ ID NO: 11-14 while retaining the HVR-L1 RASKSINKYLA (SEQ ID NO: 5), the HVR-L2 SGSTLQS (SEQ ID NO: 6), and the HVR-L3 QQHNEYPLT (SEQ ID NO: 7).

The antibody may be of the IgG4 class. The sequence of IgG4 heavy chain is

| (SEQ ID NO: 41) | |
|---|---|
| 1 | ASTKGPSVFP LAPCSRSTSE STAALGCLVK DYFPEPVTVS WNSGALTSGV HTFPAVLQSS |
| 61 | GLYSLSSVVT VPSSSLGTKT YTCNVDHKPS NTKVDKRVES KYGPPCPSCP APEFLGGPSV |
| 121 | FLFPPKPKDT LMISRTPEVT CVVVDVSQED PEVQFNWYVD GVEVHNAKTK PREEQFNSTY |
| 181 | RVVSVLTVLH QDWLNGKEYK CKVSNKGLPS SIEKTISKAK GQPREPQVYT LPPSQEEMTK |
| 241 | NQVSLTCLVK GFYPSDIAVE WESNGQPENN YKTTPPVLDS DGSFFLYSRL TVDKSRWQEG |
| 301 | NVFSCSVMHE ALHNHYTQKS LSLSLGK |

The domains of IgG4 are as follow: CH1: 1-98, Hinge: 99-110, CH2: 111-220, and CH3: 221-327. IgG4 may comprise mutations. For example, S108P mutation (for IgG4 arm swapping), L115E mutation (for FcR binding), T246W mutation (for knob in hole mutation), T246S mutation (for knob in hole mutation), L248A mutation (for knob in hole mutation), Y187V mutation (for knob in hole mutation), and/or N187A (aglycosylated for FcR binding), N187Q (aglycosylated for FcR binding), or N187G (aglycosylated for FcR binding).

One heavy chain of the single-arm antibody (the heavy chain 1 domain) of the single-arm antibody may comprise the following amino acid sequence (SEQ ID NO:2):

(SEQ ID NO: 2)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGERDSTEVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLWCLVKGFYPS
DIAVEWESNGQPENNYKTTPPVLDSDGSFFLYSRLTVDKSRWQEGNV
FSCSVMHEALHNHYTQKSLSLSLGK

The complementarity determining regions (CDRs) of SEQ ID NO: 2 are depicted in bolded and underlined text. The knob in hole T366W mutation (corresponding to IgG4 T246W mutation) in SEQ ID NO: 2 is depicted in underlined text. The S241P (for IgG4 arm swapping, corresponding to S108P) and L248E (for FCR, corresponding to L115E mutation) mutations are depicted in bolded text. In some embodiments, the HVR-H1 of the heavy chain variable domain has the sequence GYHFTSYWMH (SEQ ID NO:9), the HVR-H2 of the heavy chain variable domain has the sequence VIHPNSGSINYNEKFES (SEQ ID NO:10), and the HVR-H3 of the heavy chain variable domain has the sequence ERDSTEVLPMDY (SEQ ID NO:11).

One heavy chain of the single-arm antibody (the heavy chain 1 domain) of the single-arm antibody may comprise the following amino acid sequence (SEQ ID NO:20):

(SEQ ID NO: 20)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGERDSTEVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLWCLVKGFYPS
DIAVEWESNGQPENNYKTTPPVLDSDGSFFLYSRLTVDKSRWQEGNV
FSCSVMHEALHNHYTQKSLSLSLGK

The complementarity determining regions (CDRs) of SEQ ID NO: 20 are depicted in bolded and underlined text. The knob in hole T366W mutation (corresponding to IgG4 T246W mutation) in SEQ ID NO: 20 is depicted in underlined text. The S241P (for IgG4 arm swapping, corresponding to S108P) mutation and L248 (corresponding to L115E mutation) are depicted in bolded text.

The antibody may be of the IgG1 class. The sequence of IgG1 heavy chain is

| (SEQ ID NO: 25) | |
|---|---|
| 1 | ASTKGPSVFP LAPSSKSTSG GTAALGCLVK DYFPEPVTVS WNSGALTSGV HTFPAVLQSS |
| 61 | GLYSLSSVVT VPSSSLGTQT YICNVNHKPS NTKVDKKVEP KSCDKTHTCP PCPAPELLGG |
| 121 | PSVFLFPPKP KDTLMISRTP EVTCVVVDVS HEDPEVKFNW YVDGVEVHNA KTKPREEQYN |
| 181 | STYRVVSVLT VLHQDWLNGK EYKCKVSNKA LPAPIEKTIS KAKGQPREPQ VYTLPPSRDE |
| 241 | LTKNQVSLTC LVKGFYPSDI AVEWESNGQP ENNYKTTPPV LDSDGSFFLY SKLTVDKSRW |
| 301 | QQGNVFSCSV MHEALHNHYT QKSLSLSPGK |

The domains of IgG1 are as follow: CH1: 1-98, Hinge: 99-110, CH2: 111-223, and CH3: 224-330. IgG1 may comprise mutations. For example, L117A mutation (for FcR binding), L118A mutation (for FcR binding), T249W mutation (for knob in hole mutation), T249S mutation (for knob in hole mutation), L251A mutation (for knob in hole mutation), and/or Y290V mutation (for knob in hole mutation).

One heavy chain of the single-arm antibody (the heavy chain 1 domain) of the single-arm antibody may comprise the following amino acid sequence (SEQ ID NO: 21):

(SEQ ID NO: 21)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGERDSTEVLPMDYWGQGTTVTVSSASTKGPSVFPLAPSSKSTSG
GTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPA
PEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSN
KALPAPIEKTISKAKGQPREPQVYTLPPSRDELTKNQVSLWCLVKGF
YPSDIAVEWESNGQPENNYKTTPPVLDSDGSFFLYSKLTVDKSRWQQ
GNVFSCSVMHEALHNHYTQKSLSLSPGK

The complementarity determining regions (CDRs) of SEQ ID NO: 21 are depicted in bolded and underlined text. The knob in hole T366W mutation (corresponding to IgG1 T249W mutation) in SEQ ID NO: 21 is depicted in underlined text. The L234A (corresponding to IgG1 L117A mutation) and L235A (corresponding to IgG1 L1178 mutation) mutations are depicted in bolded text.

The heavy chain 1 of the single-arm antibody may comprise the following heavy chain variable domain amino acid sequence:

(SEQ ID NO: 15)
QVQLQQPGAELVKPGASVKLSCKSSGYHFTSYWMHWVKQRPGQGLEW
IGVIHPNSGSINYNEKFESKATLTVDKSSSTAYMQLSSLTSEDSAVY
YCAGERDSTEVLPMDYWGQGTSVTVSS.

The single-arm antibody may comprise a heavy chain variable domain amino acid sequence that is at least 85%, 90%, or 95% identical to SEQ ID NO: 15, preferably while retaining the HVR-H1 GYHFTSYWMH (SEQ ID NO: 9), the HVR-H2 VIHPNSGSINYNEKFES (SEQ ID NO: 10), and the HVR-H3 ERDSTEVLPMDY (SEQ ID NO: 11).

The single-arm antibody may comprise the following amino acid sequence of heavy chain variable domain variant 1 (VH1):

(SEQ ID NO: 16)
QVQLVQSGAELKKPGASVKVSCKSSGYHFTSYWMHWVKQAPGQGLEW
IGVIHPNSGSINYNEKFESKATITVDKSTSTAYMQLSSLTSEDSAVY
YCAGERDSTEVLPMDYWGQGTSVTVSS.

The hyper variable regions (HVRs) of VH1 are depicted in bolded and underlined text.

The single-arm antibody may comprise the following amino acid sequence of heavy chain variable domain variant 2 (VH2):

(SEQ ID NO: 17)
QVQLVQSGAELKKPGASVKVSCKSSGYHFTSYWMHWVKQAPGQGLEW
IGVIHPNSGSINYNEKFESRATITVDKSTSTAYMELSSLRSEDTAVY
YCAGERDSTEVLPMDYWGQGTTVTVSS.

The hyper variable regions (HVRs) of VH2 are depicted in bolded and underlined text.

The single-arm antibody may comprise the following amino acid sequence of heavy chain variable domain variant 3 (VH3):

(SEQ ID NO: 18)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGERDSTEVLPMDYWGQGTTVTVSS.

The hyper variable regions (HVRs) of VH3 are depicted in bolded and underlined text.

The single-arm antibody may comprise the following amino acid sequence of heavy chain variable domain variant 4 (VH4):

(SEQ ID NO: 19)
QVQLVQSGAELKKPGASVKVSCKSSGYHFTSYWMHWVRQAPGQGLEW
IGVIHPNSGSINYNEKFESRVTITVDKSTSTAYMELSSLRSEDTAVY
YCAGERDSTEVLPMDYWGQGTTVTVSS.

The hyper variable regions (HVRs) of VH4 are depicted in bolded and underlined text.

The single-arm antibody may comprise a heavy chain variable domain amino acid sequence that is at least 85%, 90%, or 95% identical to SEQ ID NO: 16-19 while retaining the HVR-H1 GYHFTSYWMH (SEQ ID NO: 9), the HVR-H2 VIHPNSGSINYNEKFES (SEQ ID NO: 10), and the HVR-H3 ERDSTEVLPMDY (SEQ ID NO: 11).

A second heavy chain of the single-arm antibody (the heavy chain 2 domain) of the single-arm antibody, which is N-terminally truncated, may comprise the following amino acid sequence (SEQ ID NO: 3):

(SEQ ID NO: 3)
ESKYGPPCPPCPAPEFEGGPSVFLFPPKPKDTLMISRTPEVTCVVVD
VSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMT
KNQVSLSCAVKGFYPSDIAVEWESNGQPENNYKTTPPVLDSDGSFFL
VSRLTVDKSRWQEGNVFSCSVMHEALHNHYTQKSLSLSLGK

There is no heavy chain variable domain and no CDRs in SEQ ID NO: 3. The knob in hole T366S/L368A/Y407V mutations in SEQ ID NO: 3 are depicted in underlined text. The S241P and L248E mutations are depicted in bolded text.

A second heavy chain of the single-arm antibody (the heavy chain 2 domain) of the single-arm antibody, which is N-terminally truncated, may comprise the following amino acid sequence (SEQ ID NO: 42):

(SEQ ID NO: 42)
ESKYGPPCPPCPAPEFLGGPSVFLFPPKPKDTLMISRTPEVTCVVVD
VSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMT
KNQVSLSCAVKGFYPSDIAVEWESNGQPENNYKTTPPVLDSDGSFFL
VSRLTVDKSRWQEGNVFSCSVMHEALHNHYTQKSLSLSLGK

A second heavy chain of the single-arm antibody (the heavy chain 2 domain) of the single-arm antibody, which is N-terminally truncated, may comprise the following amino acid sequence (SEQ ID NO: 43):

(SEQ ID NO: 43)
DKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWL
NGKEYKCKVSNKALPAPIEKTISKAKGQPREPQVYTLPPSRDELTKN
QVSLSCAVKGFYPSDIAVEWESNGQPENNYKTTPPVLDSDGSFFLVS
KLTVDKSRWQQGNVFSCSVMHEALHNHYTQKSLSLSPGK

There is no heavy chain variable domain and no CDRs in SEQ ID NO: 43. The knob in hole T366S/L368A/Y407V mutations in SEQ ID NO: 43 are depicted in underlined text. The L234A and L235A mutations are depicted in bolded text.

A second heavy chain of the antibody (the heavy chain 2 domain) may comprise any one of the following amino acid sequences (SEQ ID NOs: 44-47):

(SEQ ID NO: 44)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGKRKSTKVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK
(SEQ ID NO: 45)
QVQLVQSGAELKKPGASVKVSCKSSGYHFTSYWMKWVKQAPGQGLEW
IGVIHPNSGSINYNKKFESRVTITVDKSTSTAYMELSSLRSEDTAVY
YCAGARKSTEVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK
(SEQ ID NO: 46)
QVQLVQSGAELKKPGASVKVSCKSSGYHFTSYWMHWVKQAPGQGLEW
IGVIHPNSGSINYNKKFKSRVTITVDKSTSTAYMELSSLRSEDTAVY
YCAGKRKSTKVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK
(SEQ ID NO: 47)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGARASTAVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
EGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK

A second heavy chain of the antibody (the heavy chain 2 domain) may comprise any one of the following amino acid sequences (SEQ ID NOs: 48-51):

(SEQ ID NO: 48)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGKRKSTKVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK
(SEQ ID NO: 49)
QVQLVQSGAELKKPGASVKVSCKSSGYHFTSYWMKWVKQAPGQGLEW
IGVIHPNSGSINYNKKFESRVTITVDKSTSTAYMELSSLRSEDTAVY
YCAGARKSTEVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK
(SEQ ID NO: 50)
QVQLVQSGAELKKPGASVKVSCKSSGYHFTSYWMHWVKQAPGQGLEW
IGVIHPNSGSINYNKKFKSRVTITVDKSTSTAYMELSSLRSEDTAVY
YCAGKRKSTKVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK
(SEQ ID NO: 51)
QVQLVQSGAELKKPGASVKVSCKSSGYHFTSYWMHWVKQAPGQGLEW
IGVIHPNSGSINYNEKFESRVTITVDKSTSTAYMELSSLRSEDTAVY
YCAGARASTAVLPMDYWGQGTTVTVSSASTKGPSVFPLAPCSRSTSE
STAALGCLVKDYFPEPVTVSWNSGALTSGVHTFPAVLQSSGLYSLSS
VVTVPSSSLGTKTYTCNVDHKPSNTKVDKRVESKYGPPCPPCPAPEF
LGGPSVFLFPPKPKDTLMISRTPEVTCVVVDVSQEDPEVQFNWYVDG
VEVHNAKTKPREEQFNSTYRVVSVLTVLHQDWLNGKEYKCKVSNKGL
PSSIEKTISKAKGQPREPQVYTLPPSQEEMTKNQVSLSCAVKGFYPS
DIAVEWESNGQPENNYKTTPPVLDSDGSFFLVSRLTVDKSRWQEGNV
FSCSVMHEALHNHYTQKSLSLSLGK

The CDRs in the second heavy chain variable domain are mutated in SEQ ID NOs: 44-51 to prevent binding to C1q. The CDR mutations are depicted in bolded and underlined text. The knob in hole T366S/L368A/Y407V mutations in SEQ ID NOs: 44-51 are depicted in underlined text.

In some embodiments, the antibody that binds to C1q, comprising:
a light chain domain comprising the amino acid sequence of SEQ ID NO: 40;
a first heavy chain domain comprising the amino acid sequence of SEQ ID NO: 2; and
a second heavy chain domain comprising the amino acid sequence of SEQ ID NO: 3; the second heavy chain domain is an N-terminally truncated heavy chain.

Nucleic acids, vectors and host cells Antibodies suitable for use in the methods of the present disclosure may be produced using recombinant methods and compositions, e.g., as described in U.S. Pat. No. 4,816,567. In some embodiments, isolated nucleic acids having a nucleotide sequence encoding any of the antibodies of the present disclosure are provided. Such nucleic acids may encode an amino acid sequence containing the V_{L}/C_{L} and/or an amino acid sequence containing the V_{H}/C_{H}1 of the anti-C1q antibody. In some embodiments, one or more vectors (e.g., expression vectors) containing such nucleic acids are provided. A host cell containing such nucleic acid may also be provided. The host cell may contain (e.g., has been transduced with): (1) a vector containing a nucleic acid that encodes an amino acid sequence containing the V_{L}/C_{L} of the antibody and an amino acid sequence containing the V_{H}/C_{H}1 of the antibody, or (2) a first vector containing a nucleic acid that encodes an amino acid sequence containing the V_{L}/C_{L} of the antibody and a second vector containing a nucleic acid that encodes an amino acid sequence containing the V_{H}/C_{H}1 of the antibody. In some embodiments, the host cell is eukaryotic, e.g., a Chinese Hamster Ovary (CHO) cell or lymphoid cell (e.g., Y0, NS0, Sp20 cell). In some embodiments, the host cell is a bacterium such as E. coli.

Methods of making an anti-C1q antibody are disclosed herein. The method includes culturing a host cell of the present disclosure containing a nucleic acid encoding the anti-C1q antibody, under conditions suitable for expression of the antibody. In some embodiments, the antibody is subsequently recovered from the host cell (or host cell culture medium).

For recombinant production of a humanized anti-C1q antibody of the present disclosure, a nucleic acid encoding the antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors containing a nucleic acid sequence encoding any of the antibodies of the present disclosure, or fragments thereof polypeptides (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Stratagene, and Invitrogen.

The vectors containing the nucleic acids of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell. In some embodiments, the vector contains a nucleic acid containing one or more amino acid sequences encoding an anti-C1q antibody of the present disclosure.

Suitable host cells for cloning or expression of antibody-encoding vectors include prokaryotic or eukaryotic cells. For example, an anti-C1q antibody of the present disclosure may be produced in bacteria, in particular when glycosylation and Fc effector function are not needed. For expression of antibody fragments and polypeptides in bacteria (e.g., U.S. Pat. Nos. 5,648,237, 5,789,199, and 5,840,523; and Charlton, Methods in Molecular Biology, Vol. 248 (B. K. C. Lo, ed., Humana Press, Totowa, NJ, 2003), pp. 245-254, describing expression of antibody fragments in E. coli). In other embodiments, the antibody of the present disclosure may be produced in eukaryotic cells, e.g., a Chinese Hamster Ovary (CHO) cell or lymphoid cell (e.g., Y0, NS0, Sp20 cell) (e.g., U.S. patent application Ser. No. 14/269,950, U.S. Pat. No. 8,981,071, Eur J Biochem. 1991 Jan. 1; 195(1):235-42). After expression, the antibody may be isolated from the bacterial cell paste in a soluble fraction and can be further purified.

### Pharmaceutical Compositions, Formulations and Administration

The present disclosure is generally directed to pharmaceutical compositions comprising anti-C1q antibodies disclosed herein, including antibody Fab fragments and antibody derivatives. As used herein, the expression “pharmaceutical composition (also referred to as a “pharmaceutical formulation”) means a combination of at least one active ingredient (e.g., an anti-C1q antibody disclosed herein, including antibody Fab fragments and antibody derivatives), and at least one inactive ingredient which, when combined with the active ingredient and/or one or more additional inactive ingredients, is suitable for therapeutic administration to a human or non-human animal.

Formulations of the anti-C1q antibody (including antibody Fab fragments and antibody derivatives) were identified. Such formulations were isotonic in nature and formulated in a high concentration (e.g., greater than about 75 mg/ml). In certain embodiments, the formulations exhibited high levels of stability. Such formulations are also able to meet Subvisible Particulate Matter USP<789> criteria for ophthalmology products. An example of a selected formulation is at least 75 mg/ml (for example, 100 mg/mL, 125 mg/mL, 150 mg/mL, 175 mg/mL, 200 mg/mL, 225 mg/mL, 250 mg/mL anti-C1q antibody (or antibody Fab fragments and antibody derivatives)) formulated in 5 to 25 mM sodium citrate or sodium succinate (for example, 5 mM, 10 mM, 15 mM, 20, mM, 25 mM sodium citrate or sodium succinate), 3% to 7% trehalose (for example, 3%, 4%, 5%, 6%, 7% (w/v) trehalose), 0.04% to 0.06% polysorbate 20 or polysorbate 80 (for example, 0.04%, 0.05%, 0.06% (w/v) polysorbate 20 (PS20) or polysorbate 80 (PS80)), and pH 6.4-7.5 (for example, 6.4, 6.6. 6.8. 7.0, 7.2, 7.4). The pharmaceutical composition may further comprise 30 mM to 50 mM NaCl (for example, 30 mM, 40 mM, 50 mM NaCl).

The pharmaceutical composition may comprise sodium citrate or sodium succinate buffer. The sodium citrate or sodium succinate may be about 5 mM to about 25 mM. In some embodiments, the sodium citrate or sodium succinate may be about 5 mM to about 20 mM. In some embodiments, the sodium citrate or sodium succinate may be about 5 mM to about 10 mM. In some embodiments, the sodium citrate may be about 10 mM. In some embodiments, the sodium succinate may be about 10 mM.

The pharmaceutical composition may further comprise a stabilizer such as trehalose, which reduces osmolality and keeps the composition isotonic. The trehalose may be between about 3% to about 7% (w/v). In some embodiments, the trehalose may be 3%, 4%, 5%, 6% or 7% (w/v). In some embodiments, the trehalose may be 5% (w/v).

The pharmaceutical composition may further comprise a surfactant such as PS20 or PS80 to minimize aggregation (subvisible particle reduction). The PS20 or PS80 may be 0.04%-0.06%. In some embodiments, the PS20 may be 0.06% (w/v). In some embodiments, the PS80 may be 0.06% (w/v).

The pharmaceutical composition may be at a pH of 6.4-7.5. In some embodiments, the pharmaceutical composition may have a pH of about 7.2.

Optionally, the pharmaceutical composition may further comprise 30 mM to 50 mM NaCl. In some embodiments, the pharmaceutical composition may optionally comprise 40 mM NaCl.

In certain aspects, provided herein is a pharmaceutical composition comprising: (a) at least 75 mg/ml of an anti-C1q antibody; from 5 to 25 mM sodium citrate or sodium succinate; (c) from 3% to 7% trehalose; and (d) from 0.04% to 0.06% polysorbate 20 or polysorbate 80, wherein the pharmaceutical composition is at a pH of 6.4-7.5.

In certain embodiments, the pharmaceutical composition comprises at least 75 mg/ml, at least 100 mg/ml, at least 125 mg/ml, at least 150 mg/ml, at least 175 mg/ml, at least 200 mg/ml, at least 225 mg/ml, or at least 250 mg/ml of the anti-C1q antibody, including antibody Fab fragments and antibody derivatives. The pharmaceutical composition may comprise from 75 mg/ml to 300 mg/ml, from 100 mg/ml to 300 mg/ml, or from 125 mg/ml to 250 mg/ml of the anti-C1q antibody, including antibody Fab fragments and antibody derivatives. In some embodiments, the pharmaceutical composition may comprise about 75 mg/ml, 100 mg/ml, 125 mg/ml, about 150 mg/ml, about 175 mg/ml, about 200 mg/ml, 225 mg/ml, or about 250 mg/ml of the anti-C1q antibody Fab fragment. The pharmaceutical composition may comprise 100 mg/ml to 125 mg/ml, 125 mg/ml to 150 mg/ml, 150 mg/ml to 175 mg/ml, 175 mg/ml to 200 mg/ml, or 200 mg/ml to 250 mg/ml of the anti-C1q antibody, including antibody Fab fragments and antibody derivatives.

In certain embodiments, the pharmaceutical composition comprises about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM sodium citrate or sodium succinate. The sodium citrate may be 10 mM. The sodium succinate may be 5 mM to 20 mM. The pharmaceutical composition may comprise 5 mM to 10 mM, 10 mM to 15 mM, 15 mM to 20 mM, or 20 mM to 25 mM sodium citrate or sodium succinate.

In certain embodiments, the pharmaceutical composition comprises about 3%, 4%, 5%, 6%, or 7% a stabilizer. The pharmaceutical composition may comprise 3%-4%, 4%-5%, 5%-6%, or 6%-7% trehalose. The stabilizer such as trehalose increases osmolality. The trehalose may be present at an amount of about 4% to about 6%, preferably about 5% (w/v).

In certain embodiments, the pharmaceutical composition comprises about 0.04%, 0.05%, or 0.06% surfactant to minimize aggregation (subvisible particle reduction). The surfactant may be polysorbate 20 (PS20) or polysorbate 80 (PS80). The pharmaceutical composition may comprise 0.04% to 0.05% or 0.05% to 0.06% polysorbate 20 or polysorbate 80.

In certain embodiments, the pharmaceutical composition is at a pH of about 6.4, about 6.5, about 6.6, about 6.7, about 6.8, about 6.9, about 7.0, about 7.1, about 7.2, about 7.3, about 7.4, or about 7.5. The pharmaceutical composition may be at a pH of 6.0-6.5 or 6.5-7.0 or 7.0 to 7.5.

Optionally, the pharmaceutical composition further comprises from 30 mM to 50 mM NaCl. The pharmaceutical composition may comprise about 30 mM, about 35 mM, about 40 mM, about 45 mM, or about 50 mM NaCl. NaCl may be added for isotonicity. The pharmaceutical composition may comprise 30 mM to 35 mM, 35 mM to 40 mM, 40 mM to 45 mM, or 45 mM to 50 mM NaCl. The amount of NaCl may depend on amount of trehalose in formulation. The more trehalose added, the less NaCl is needed. The amount of NaCl may also depend on antibody concentration.

In certain embodiments, the viscosity of the pharmaceutical composition is no more than 10 cP at 25° C. The viscosity of the pharmaceutical composition may be about 1 cP, about 2 cP, about 3 cP, about 4 cP, about 5 cP, about 6 cP, about 7 cP, about 8 cP, about 9 cP, or about 10 cP at 25° C. The viscosity of the pharmaceutical composition may be 1 cp to 5 cp or 5 cp to 10 cp at 25° C.

The pharmaceutical composition may be stable for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, or at least 24 weeks. The pharmaceutical composition may be stable at a temperature equal to or greater than 25° C. In some embodiments, the pharmaceutical composition may be stable at a temperature of between about 25° C. to about 40° C. The pharmaceutical composition may be stable at a temperature of about 25° C., about 30° C., about 35° C., or about 40° C.

In some embodiments, the pharmaceutical composition is stable for a minimum of at least 1 year, at least 1.5 years, or at least two years. The pharmaceutical composition may be stable at a temperature of from −20° C. to 25° C. The pharmaceutical composition may be stable at a temperature of about −20° C., about −15° C., about −10° C., about −5° C., about 0° C., about 2° C., about 4° C., about 5° C., about 8° C., about 10° C., about 15° C., about 20° C., or about 25° C. The pharmaceutical composition may be stable at a temperature of from 2° C. to 8° C. The pharmaceutical composition may be stable at a temperature of greater than or equal to 25° C., such as between about 25° C. to about 40° C., preferably about 25° C., about 30° C., about 35° C., or about 40° C.

The pharmaceutical composition may be stable after at least one, at least two, at least three, at least four, or at least five freeze/thaw cycles. The pharmaceutical composition may be stable after at least one, at least two, or at least three days of continuous agitation.

In certain embodiments, the pharmaceutical composition is prepared for intravitreal or intraocular injection. The pharmaceutical composition may be contained in a syringe. In some embodiments, the pharmaceutical composition is prefilled in a syringe. In some embodiments, the syringe has a fill volume of no more than 300 microliter. In some embodiments, the syringe delivers a volume of 25-100 microliter.

In certain embodiments, the pharmaceutical composition is prepared for subcutaneous injection. The pharmaceutical composition may be contained in an injector, autoinjector, or pen. In some embodiments, the injector, autoinjector, or pen has a fill volume of no more than 10 ml.

In certain embodiments, the pharmaceutical composition is prepared for intravenous injection. The pharmaceutical composition may be contained in an intravenous solution bag.

In certain embodiments, the pharmaceutical compositions provided herein exhibit high levels of stability. The term “stable,” as used herein in reference to the pharmaceutical compositions, means that the antibodies within the pharmaceutical compositions retain an acceptable degree of structure and/or function and/or biological activity after storage for a defined amount of time. A composition may be stable even though the antibody contained therein does not maintain 100% of its structure and/or function and/or biological activity after storage for a defined amount of time. Under certain circumstances, maintenance of about 80%, about 85%, about 90%, about 95%, about 96%, about 97%, about 98% or about 99% of an antibody's structure and/or function and/or biological activity after storage for a defined amount of time may be regarded as “stable.”

Stability can be measured, inter alia, by determining the percentage of native antibody remaining in the composition after storage for a defined amount of time at a given temperature. The “native antibody” refers to an antibody with a “native conformation,” which refers to the secondary, tertiary and/or quaternary structures of the antibody in its biologically active state. For example, the native antibody is in a monomer form. Several methods based on physical parameters are available for analyzing and quantitating aggregated antibodies and determining amounts of aggregated antibodies and monomeric antibodies. The percentage of native or monomer antibody can be determined by, inter alia, size exclusion chromatography (e.g., size exclusion high performance liquid chromatography [SE-HPLC]). An “acceptable degree of stability,” as that phrase is used herein, means that at least 90% of the native or monomer form of the antibody can be detected in the composition after storage for a defined amount of time at a given temperature. In certain embodiments, at least about 90%, 91%, 92%, 93%, 94%, 95%, 96%, 97%, 98%, 99% or 100% of the native or monomer form of the antibody can be detected in the composition after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, or more. The pharmaceutical composition may be stable for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks, at least 17 weeks, at least 18 weeks, at least 19 weeks, at least 20 weeks, at least 21 weeks, at least 22 weeks, at least 23 weeks, or at least 24 weeks. The pharmaceutical composition may be stable for a minimum of two years. The temperature at which the pharmaceutical composition may be stored when assessing stability can be any temperature from about −20° C. to 25° C., e.g., storage at about −20° C., about −15° C., about −10° C., about −5° C., about 0° C., about 2° C., about 4° C., about 5° C., about 8° C., about 10° C., about 15° C., about 20° C., or about 25° C. The pharmaceutical composition may be stable at a temperature of from 2° C. to 8° C. The pharmaceutical composition may be stable at a temperature of greater than or equal to 25° C., such as between about 25° C. to about 40° C., preferably about 25° C., about 30° C., about 35° C., or about 40° C. For example, a pharmaceutical composition may be deemed stable if after 3 months of storage at 5° C., greater than about 90%, 95%, 96% or 97% of native or monomer antibody is detected by SE-HPLC. A pharmaceutical composition may also be deemed stable if after 6 months of storage at 5° C., greater than about 90%, 95%, 96% or 97% of native or monomer antibody is detected by SE-HPLC. A pharmaceutical composition may also be deemed stable if after 9 months of storage at 5° C., greater than about 90%, 95%, 96% or 97% of native or monomer antibody is detected by SE-HPLC. A pharmaceutical composition may also be deemed stable if after 3 months of storage at 25° C., greater than about 90%, 95%, 96% or 97% of native or monomer antibody is detected by SE-HPLC. A pharmaceutical composition may also be deemed stable if after 6 months of storage at 25° C., greater than about 90%, 95%, 96% or 97% of native or monomer antibody is detected by SE-HPLC. A pharmaceutical composition may also be deemed stable if after 9 months of storage at 25° C., greater than about 90%, 95%, 96% or 97% of native or monomer antibody is detected by SE-HPLC.

Other methods may be used to assess the stability of the pharmaceutical compositions disclosed herein, such as, SEC-HPLC, visual particle inspection, and light obscuration by HIAC for measurement of aggregation, visible particles, and subvisible particles, respectively. Another stability-indicating method is cation exchange HPLC, which was also used to detect changes in charge variants profile for stability assessment.

Stability may also be assessed by measuring the biological activity and/or binding affinity of the antibody to its target. For example, a composition disclosed herein may be regarded as stable if, after storage at e.g., 5° C., 250 C, 40° C., etc. for a defined amount of time (e.g., 1 to 12 months), the anti-C1q antibody contained within the composition binds to C1q with an affinity that is at least 50%, 60%, 70%, 80%, 90%, 95%, or more of the binding affinity of the anti-C1q antibody contained within the composition prior to said storage. Additional methods for assessing the stability of an antibody in composition are demonstrated in the Examples presented below.

The stability of the pharmaceutical composition may be assessed according to the evolution in time of its physical and chemical parameters. Physical stability includes visual and olfactory data together with the tracing of invisible particles. To study the chemical stability, an analysis method is used to achieve a separation and even a quantification of any degradation products. Monitoring of the concentration in active principle and the appearance of degradation products with time is completed using methods known to one of ordinary skill in the art, including by, for example, UV280 (for concentration), and SEC-HPLC, sodium dodecyl sulfate capillary gel electrophoresis (CE-SDS), and cation exchange HPLC (for appearance of degradation products). The pharmaceutical composition may be stable if compliant with USP<789>, USP<787>, or USP<788> limits.

As used herein, a low level of viscosity, in reference to a pharmaceutical composition disclosed herein, will exhibit an absolute viscosity of less than about 20 cPoise (cP) at 25° C. For example, a pharmaceutical composition disclosed herein will be deemed to have “low viscosity,” if, when measured using standard viscosity measurement techniques, the composition exhibits an absolute viscosity of about 19 cP, about 18 cP, about 17 cP, about 16 cP, about 15 cP, about 14 cP, about 13 cP, about 12 cP, about 11 cP, about 10 cP, about 9 cP, about 8 cP, about 7 cP, about 6 cP, about 5 cP, about 4 cP, or less at 25° C. As used herein, a moderate level of viscosity, in reference to a pharmaceutical composition disclosed herein, will exhibit an absolute viscosity of between about 30 cP and about 20 cP at 25° C. For example, a pharmaceutical composition disclosed herein will be deemed to have “moderate viscosity,” if when measured using standard viscosity measurement techniques, the composition exhibits an absolute viscosity of about 30 cP, about 29 cP, about 28 cP, about 27 cP, about 26 cP, about 25 cP, about 24 cP, about 23 cP, about 22 cP, about 21 cP or about 20 cP at 25° C.

In some embodiments, the osmolality of the pharmaceutical composition is within physiological osmolality range of 250-400 mOsm/kg. In some embodiments, the viscosity of the pharmaceutical composition is no more than 30 cP at 25° C. In some embodiments, the viscosity of the pharmaceutical composition is no more than 15 cP at 25° C. In some embodiments, the viscosity of the pharmaceutical composition is about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, about 26 cP, about 27 cP, about 28 cP, about 29 cP, or about 30 cP at 25° C.

### Administration

Disclosed herein are methods of administering a pharmaceutical composition comprising at least 75 mg/ml of an anti-C1q antibody. The pharmaceutical composition may further comprise from 5 to 25 mM sodium citrate or sodium succinate; from 3% to 7% trehalose; and/or from 0.04% to 0.06% polysorbate 20 or polysorbate 80. The pharmaceutical composition may be at a pH of 6.4-7.5. The anti-C1q antibody may comprise a light chain variable domain and a heavy chain variable domain. The antibody may bind to at least human C1q, mouse C1q, or rat C1q. The antibody may be a humanized antibody, a chimeric antibody, or a human antibody. The antibody may be a monoclonal antibody, an antibody fragment thereof, and/or an antibody derivative thereof. In some embodiments, the antibody is humanized antibody. In some embodiments, the antibody is antibody fragment, such as, a Fab fragment. The light chain variable domain of the antibody, the antibody fragment thereof, and/or the antibody derivative thereof comprises the HVR-L1, HVR-L2, and HVR-L3 of the monoclonal antibody M1 produced by a hybridoma cell line deposited with Accession Number PTA-120399. The heavy chain variable domain of the antibody, the antibody fragment thereof, and/or the antibody derivative thereof comprises the HVR-H1, HVR-H2, and HVR-H3 of the monoclonal antibody M1 produced by a hybridoma cell line deposited with ATCC Accession Number PTA-120399.

In some embodiments, the amino acid sequence of the light chain variable domain and heavy chain variable domain comprise one or more of SEQ ID NO:5 of HVR-L1, SEQ ID NO:6 of HVR-L2, SEQ ID NO:7 of HVR-L3, SEQ ID NO:9 of HVR-H1, SEQ ID NO:10 of HVR-H2, and SEQ ID NO:11 of HVR-H3.

The antibody may comprise a light chain variable domain amino acid sequence that is at least 85%, 90%, or 95% identical to SEQ ID NO:4, preferably while retaining the HVR-L1 RASKSINKYLA (SEQ ID NO:5), the HVR-L2 SGSTLQS (SEQ ID NO:6), and the HVR-L3 QQHNEYPLT (SEQ ID NO:7). The antibody may comprise a heavy chain variable domain amino acid sequence that is at least 85%, 90%, or 95% identical to SEQ ID NO:8, preferably while retaining the HVR-H1 GYHFTSYWMH (SEQ ID NO:9), the HVR-H2 VIHPNSGSINYNEKFES (SEQ ID NO:10), and the HVR-H3 ERDSTEVLPMDY (SEQ ID NO:11).

In certain embodiments of the compositions and methods provided herein, the pharmaceutical composition is formulated for intravenous administration. In some embodiments, the pharmaceutical composition is administered intravenously. In certain embodiments of the compositions and methods provided herein, the pharmaceutical composition is formulated for subcutaneous administration. In some embodiments, the pharmaceutical composition is administered subcutaneously. In certain embodiments, the pharmaceutical composition is formulated for intramuscular administration. In certain embodiments, the pharmaceutical composition is administered intramuscularly. In some embodiments, the pharmaceutical composition is formulated for infraorbital, intravitreal, intraocular, subconjunctival, retrobulbar, peribulbar, and/or intrathecal administration. In certain embodiments, the pharmaceutical composition is administered by infraorbital, intravitreal, intraocular, subconjunctival, retrobulbar, peribulbar, and/or intrathecal injection.

In certain embodiments, the pharmaceutical composition is administered in a delivery volume of no more than 3 ml, 2.5 ml, 2 ml, 1.5 ml, or 1 ml. In some embodiments, the pharmaceutical composition is administered in a delivery volume of no more than 2 ml. In some embodiments, the pharmaceutical composition is administered in a delivery volume of no more than 300 microliters. In some embodiments, the pharmaceutical composition is administered in a delivery volume of no more than 25 microliters, 50 microliters, 75 microliters, or 100 microliters.

In certain aspects, the present disclosure relates to an injector comprising the pharmaceutical composition disclosed herein. The injector may be for subcutaneous, intravitreal or intraocular injection. In some embodiments, the injector comprises a delivery volume of no more than 10 μl. In some embodiments, the injector comprises a needle of a size of no bigger than 25G (25 gauge). In some embodiments, the injector is an automatic reusable fixed dose pen. In other embodiments, the injector is an automatic reusable variable dose pen. In yet other embodiments, the injector is an automatic disposable fixed dose autoinjector. In some embodiments, the injector comprises a delivery volume of no more than 300 microliters. The injector may be a syringe.

In certain aspects, the present disclosure relates to prefilled syringe comprising the pharmaceutical composition disclosed herein. The prefilled syringe may comprise a delivery volume of no more than 300 microliters.

In some embodiments, the pharmaceutical compositions provided herein may be contained within any container suitable for storage of medicines and other therapeutic compositions. For example, the pharmaceutical compositions may be contained within a sealed and sterilized plastic or glass container having a defined volume such as a vial, ampule, syringe, cartridge, or bottle. Different types of vials can be used to contain the compositions provided herein, including, e.g., clear and opaque (e.g., amber) glass or plastic vials. Likewise, any type of syringe can be used to contain and/or administer the pharmaceutical compositions disclosed herein.

The pharmaceutical compositions provided herein may be contained within plastic polymer syringes that are silicon-free and low in particulate level, i.e., compliant with USP<789> limits (less than fifty particles per ml of 10 μm particles and less than five particles per ml of 25 μm particles) for subvisible particulate matter (See USP<789>). Particulate matter in ophthalmic solutions. USP 35-NF 30. MD, USA: The United States Pharmacopeial Convention; 2012). For example, Terumo's i-coating stopper technology removes the need of silicone oil in the syringe system while providing consistent and predictable break-loose and glide forces. Eliminating the use of silicone oil in the Pre-Filled Syringe (PFS) system significantly reduces the sub-visible particle load and allows the fulfilment of stringent particle requirements such as USP<789> Particulate Matter in Ophthalmic Solutions.

The pharmaceutical compositions provided herein may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical compositions provided herein, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical compositions disclosed herein are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the methods disclosed herein are commercially available under the tradename “FluroTec®,” available from West Pharmaceutical Services, Inc. (Lionville, Pa.).

In certain embodiments, the pharmaceutical compositions can be administered to a patient by intravitreal or intraocular injection.

In certain embodiments, the pharmaceutical compositions can be administered to a patient by parenteral routes such as injection (e.g., subcutaneous, intravenous, intramuscular, intraperitoneal, infraorbital, intravitreal, intraocular, subconjunctival, retrobulbar, peribulbar, and/or intrathecal injection). Numerous reusable pen and/or autoinjector delivery devices can be used to subcutaneously deliver the pharmaceutical compositions disclosed herein.

Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™ and OPTICLIK™ (sanofi-aventis, Frankfurt, Germany), to name only a few. Examples of disposable pen and/or autoinjector delivery devices having applications in subcutaneous delivery of a pharmaceutical composition disclosed herein include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park, Ill.), to name only a few.

The use of a microinfusor to deliver the pharmaceutical compositions disclosed herein is also contemplated herein. As used herein, the term “microinfusor” means a subcutaneous delivery device designed to slowly administer large volumes (e.g., up to about 2.5 mL or more) of a therapeutic composition over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996).

### Methods of Treatment

In certain aspects, the present disclosure relates to methods of preventing, reducing risk of developing, or treating a disease or disorder associated with complement pathway comprising administering the pharmaceutical composition disclosed herein. The present disclosure is also generally directed to methods of preventing, reducing risk of developing, or treating diseases and disorders associated with complement pathway comprising administering the pharmaceutical composition using the injector, the syringe, or the intravenous solution bag disclosed herein.

The methods of the invention provide for modulating the immune response to diseases disclosed herein through administering agents that are antagonists of complement. For example, without being bound by theory, immature astrocytes induce expression of C1q proteins in neurons during development. Inflammatory mediators such as complement factor are normally expressed at very low levels in healthy brain tissue but can be rapidly induced by a variety of insults to the brain such as infection, ischaemia, and injury. Activation of C1q, C1r, and C1s contributes to the inflammatory response, which leads to synaptic loss, along with the generation and recurrence of seizures and seizure-related neuronal damage. During the developmental process of neurodegenerative disease, overexpression of C1q, C1r, and C1s may be coupled with a signal for complement activation, e.g., β-amyloid, APP, cytokines such as IFNγ, TNFα, and the like, also resulting in inflammation.

By administering the pharmaceutical composition disclosed herein that inhibits complement activation, synapses can be maintained that would otherwise be lost.

The methods neutralize complement biological activity. The affected complement biological activity could be (1) C1q binding to an autoantibody, (2) C1q binding to C1r, (3) C1q binding to C1s, (4) C1q binding to IgM, (5) C1q binding to phosphatidylserine, (6) C1q binding to pentraxin-3, (7) C1q binding to C-reactive protein (CRP), (8) C1q binding to globular C1q receptor (gC1qR), (9) C1q binding to complement receptor 1 (CR1), (10) C1q binding to beta-amyloid, (11) C1q binding to calreticulin, (12) C1q binding to apoptotic cells, or (13) C1q binding to B cells. The affected complement biological activity could further be (1) activation of the classical complement activation pathway, (2) activation of antibody and complement dependent cytotoxicity, (3) CH50 hemolysis, (4) synapse loss, (5) B-cell antibody production, (6) dendritic cell maturation, (7) T-cell proliferation, (8) cytokine production (9) microglia activation, (10) Arthus reaction, (11) phagocytosis of synapses or nerve endings, or (12) activation of complement receptor 3 (CR3/C3) expressing cells.

The methods promote improved maintenance of neuronal function in conditions associated with synapse loss. The maintenance of neural connections provides for functional improvement in neurodegenerative disease relative to untreated patients. The prevention of synapse loss may comprise at least a measurable improvement relative to a control lacking such treatment over the period of 1, 2, 3, 4, 5, 6 days or at least one week, for example at least a 10% improvement in the number of synapses, at least a 20% improvement, at least a 50% improvement, or more.

In one aspect, described herein is a method of inhibiting synapse loss comprising administering to a patient suffering from adverse synapse loss the pharmaceutical composition disclosed herein. In some embodiments, the patient has suffered synapse loss as a result of a neurodegenerative disorder, central nervous system disorder, or a peripheral nervous system disorder. In some embodiments, the method further comprises administration of neural progenitors, or a neurogenesis enhancer. In some embodiments, the antibody binds to C1q and inhibits complement activation.

In one aspect, described herein is a method of treating or preventing a disease associated with complement activation in an individual in need of such treatment, the method comprising administering the pharmaceutical composition disclosed herein. The disease associated with complement activation may be a neurodegenerative disorder, which may be associated with loss of synapses or loss nerve connections, associated with synapse loss that is dependent on the complement receptor 3(CR3)/C3 or complement receptor CR1, associated with pathological activity-dependent synaptic pruning, or associated with synapse phagocytosis by microglia. The neurodegenerative disorder may be Alzheimer's disease, amyotrophic lateral sclerosis (ALS), multiple sclerosis, an ophthalmic disorder, glaucoma, myotonic dystrophy, Guillain-Barre' syndrome (GBS), Myasthenia Gravis, Bullous Pemphigoid, spinal muscular atrophy, Down syndrome, Parkinson's disease, or Huntington's disease (HD). In some embodiments, the neurodegenerative disorder is ALS, GBS or HD.

In some embodiments, the disease associated with complement activation is an inflammatory disease, autoimmune disease, complement-associated eye disease or metabolic disorder, such as diabetes, obesity, rheumatoid arthritis (RA), acute respiratory distress syndrome (ARDS), remote tissue injury after ischemia and reperfusion, complement activation during cardiopulmonary bypass surgery, dermatomyositis, pemphigus, lupus nephritis and resultant glomerulonephritis and vasculitis, cardiopulmonary bypass, cardioplegia-induced coronary endothelial dysfunction, type II membranoproliferative glomerulonephritis, IgA nephropathy, acute renal failure, cryoglobinemia, antiphospholipid syndrome, glaucoma, Chronic open-angle glaucoma, acute closed angle glaucoma, macular degenerative diseases, age-related macular degeneration (AMD), geographic atrophy, choroidal neovascularization (CNV), uveitis, diabetic retinopathy, ischemia-related retinopathy, endophthalmitis, intraocular neovascular disease, diabetic macular edema, pathological myopia, von Hippel-Lindau disease, histoplasmosis of the eye, Neuromyelitis Optica (NMO), Central Retinal Vein Occlusion (CRVO), corneal neovascularization, retinal neovascularization, Leber's hereditary optic neuropathy, optic neuritis, Behcet's retinopathy, ischemic optic neuropathy, retinal vasculitis, ANCA vasculitis, Purtscher retinopathy, Sjogren's dry eye disease, dry AMD, wet AMD, sarcoidosis, temporal arteritis, polyarteritis nodosa, multiple sclerosis, allo-transplantation, hyperacute rejection, hemodialysis, chronic occlusive pulmonary distress syndrome (COPD), asthma, aspiration pneumonia, multiple sclerosis, Guillain-Barre syndrome, Myasthenia Gravis, Bullous Pemphigoid, or myositis.

In some embodiments, the disease associated with complement activation is an autoimmune disease selected from myasthenia gravis, Diabetes mellitus type 1, Hashimoto's thyroiditis, Addison's disease, Coeliac disease, Crohn's disease, pernicious anaemia, Pemphigus vulgaris, vitiligo, autoimmune hemolytic anemias, paraneoplastic syndromes, a vasculitis disease, hypocomplementemic urticarial vasculitis (HUV), polymyalgia rheumatica, temporal arteritis, Wegener's granulomatosis, multiple sclerosis, Guillain-Barre syndrome, Myasthenia Gravis, Bullous Pemphigoid, or myositis.

In some embodiments, the disease associated with complement activation is a blood disorder selected from cold agglutinin hemolytic anemia (cold agglutinin disease), cold antibody hemolytic anemia, ABO incompatible acute hemolytic reactions, warm agglutinin hemolytic anemia, warm antibody hemolytic anemia, warm autoimmune hemolytic anemia (WAIHA), autoimmune hemolytic anemia (AIHA) autoimmune thrombocytopenia, antiphospholipid syndrome, Evan's syndrome, neonatal alloimmune thrombocytopenia, red blood cell alloimmunization, Felty's syndrome, antibody mediated thrombocytopenia, heparin-induced thrombocytopenia (HIT), heparin-induced thrombocytopenia and thrombosis (HITT), thrombotic thrombocytopenic purpura (TTP), immune thrombocytopenic purpura (ITP), thrombocytopenia, thrombosis, vasculitis, lupus nephritis, systemic lupus erythematosus (SLE), glomerulonephritis, anti-phospholipid antibody syndrome (APS), an infection, or a drug-induced hematologic disorder.

By “neurological” or “cognitive” function as used herein, it is meant that the decrease of synapses in the brain enhances the patient's ability to think, function, etc. As used herein, the term “subject” encompasses mammals and non-mammals. Examples of mammals include, but are not limited to, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

The complement inhibitors of the present disclosure may be used, without limitation, conjointly with any additional treatment, such as immunosuppressive therapies, for any disease disclosed herein, including autoimmune and/or neurodegenerative diseases.

In some embodiments, the pharmaceutical composition disclosed herein is administered in combination with an inhibitor of the alternative pathway of complement activation. Such inhibitors may include, without limitation, factor B blocking antibodies, factor D blocking antibodies, soluble, membrane-bound, tagged or fusion-protein forms of CD59, DAF, CR1, CR2, Crry or Comstatin-like peptides that block the cleavage of C3, non-peptide C3aR antagonists such as SB 290157, Cobra venom factor or non-specific complement inhibitors such as nafamostat mesilate (FUTHAN; FUT-175), aprotinin, K-76 monocarboxylic acid (MX-1) and heparin (see, e.g., T. E. Mollnes & M. Kirschfink, Molecular Immunology 43 (2006) 107-121). In some embodiments, the pharmaceutical composition disclosed herein is administered in combination with an inhibitor of the interaction between the autoantibody and its autoantigen. Such inhibitors may include purified soluble forms of the autoantigen, or antigen mimetics such as peptide or RNA-derived mimotopes, including mimotopes of the AQP4 antigen. Alternatively, such inhibitors may include blocking agents that recognize the autoantigen and prevent binding of the autoantibody without triggering the classical complement pathway. Such blocking agents may include, e.g., autoantigen-binding RNA aptamers or antibodies lacking functional C1q binding sites in their Fc domains (e.g., Fab fragments or antibody otherwise engineered not to bind C1q).

### EXAMPLES

### Example 1: Formulation Development of the Anti-C1q Fab Fragment (Fab A)

The feasibility of high concentration anti-C1q antibody Fab fragment formulations were assessed at 200 mg/mL using two formulation candidates: IVT-1 (10 mM histidine, 5% (w/v) trehalose dihydrate, 40 mM sodium chloride, pH 6.4) and IVT-2 (10 mM sodium succinate, 500 (w/v) trehalose dihydrate, 40 mM sodium chloride, 0.0400 (w/v) Polysorbate 20, pH 6.4). Subvisible particle (SVP) count data indicate that the presence of a surfactant is required to stabilize the anti-C1q antibody Fab fragment at 200 mg/mL.

Table 1 shows subvisible particle count for the two formulation candidates.

| | | | | Particles per |
|---|---|---|---|---|
| | Protein | | | mL (10 μm / 25 |
| Formulation | Concentration | | Appearance | μm)* |
| IVT-1 | 188 | mg/mL | Clear | 2800 / 67 |
| 10 mM histidine, 5% (w/v) | | | | |
| trehalose dihydrate, 40 mM | | | | |
| sodium chloride, pH 6.4 | | | | |
| IVT-2 | 224 | mg/mL | Clear | 54 / 0 |

10 mM sodium succinate, 5%
(w/v) trehalose dihydrate, 40 mM
sodium chloride, 0.04% (w/v)
Polysorbate 20, pH 6.4

*0.2 μm filtered prior to analysis

Polysorbate 20 (PS20) surfactant was added to all formulation candidates. Sorbitol and 2-hydroxypropyl-β-cyclodextrin (2HPBCD) were evaluated as alternative for trehalose dihydrate. Further, effect of freeze/thaw (F/T) cycling (freeze at −20° C./thaw at room temperature), agitation stress, and accelerated stability were evaluated.

TABLE 2
shows composition of the formulation candidates.

| Formulation | Composition |
|---|---|
| IVT-1P | 10 mM histidine, 5% (w/v) trehalose dihydrate, 40 mM sodium |
| | chloride, 0.04% (w/v) PS20, pH 6.4 |
| IVT-2 | 10 mM sodium succinate, 5% (w/v) trehalose dihydrate, 40 mM |
| | sodium chloride, 0.04% (w/v) PS20, pH 6.4 |
| IVT-3* | 10 mM sodium succinate, 5% (w/v) 2-hydroxypropyl-β-cyclodextrin, |
| | 40 mM sodium chloride, 0.04% (w/v) PS20, pH 6.4 |
| IVT-4 | 10 mM sodium succinate, 5% (w/v) sorbitol, 40 mM sodium |
| | chloride, 0.04% (w/v) PS20, pH 6.4 |

*IVT-3 formulation sample showed gel like appearance upon storage at 5° C.

All samples were clear with no visible particles at t=0.

TABLE 3
shows characteristics of formulation candidates at t = 0.

| Test | IVT-1P | IVT-2 | IVT-4 |
|---|---|---|---|
| Concentration, | 197 | 178 | 196 |
| mg/mL | | | |
| Visible Particulates | Clear and No Visible | Clear and No Visible | Clear and No Visible |
| | Particles | Particles | Particles |
| Clarity and | Clear | Clear | Clear |
| Opalescence | Btw. Std I-II | Btw. Std II-III | Eqv. Std II |
| Viscosity, cP at | 12.0 | 7.11 | 8.46 |
| 25° C. | | | |
| pH | 6.61 | 6.59 | 6.44 |
| Osmolality, | 340 | 344 | 480 |
| mOSmo/kg | | | |
| Sub Visible | 18/7 | 23/4 | 83/56 |
| Particles, particles | | | |
| per mL, 10 μm/25 | | | |
| μm | | | |
| SE-HPLC | Main: 98.3% | Main: 98.8% | Main: 98.7% |
| | HMW: 1.2% | HMW: 1.2% | HMW: 1.2% |
| | LMW: 0.5% | LMW: 0.0% | LMW: 0.1% |
| CE-SDS, reducing | HC + LC: 100% | HC + LC: 100% | HC + LC: 100% |
| CE-SDS, non- | Intact: 100% | Intact: 100% | Intact: 100% |
| reducing | | | |
| CEX-HPLC | Peak 1 + | Peak 1 + | Peak 1 + |
| | Peak 2: 93.2% | Peak 2: 95.1% | Peak 2: 95.0% |
| | Pre: 5.2% | Pre: 3.7% | Pre: 3.7% |
| | Inter: 1.0% | Inter: 0.7% | Inter: 0.8% |
| | Post: 0.1% | Post: 0.1% | Post: 0.1% |

Samples were hazy and failed SVP specifications per USP<789> after 5 freeze/thaw (F/T) cycles. Further, chemical stability after 5 F/T cycles was unchanged for all formulation candidates.

TABLE 4
shows characteristics after 5 freeze/thaw cycles.

| Test | IVT-1P | IVT-2 | IVT-4 |
|---|---|---|---|
| Concentration, | 200 | 179 | 196 |
| mg/mL | | | |
| Visible Particulates | Few particles | Few particles | Few particles |
| Clarity and | Least hazy | Less hazy | Less hazy |
| Opalescence | Eqv. Std II | Eqv. Std II | Btw. Std II-III |
| Sub Visible | 165/46 | 91/13 | 155/25 |
| Particles, particles | | | |
| per mL, 10 μm/25 | | | |
| μm | | | |
| SE-HPLC | Main: 98.4% | Main: 98.8% | Main: 98.7% |
| | HMW: 1.2% | HMW: 1.1% | HMW: 1.3% |
| | LMW: 0.4% | LMW: 0.0% | LMW: 0.1% |
| CE-SDS, reducing | HC + LC: 100% | HC + LC: 100% | HC + LC: 100% |
| CE-SDS, non- | Intact: 99% | Intact: 99% | Intact: 99% |
| reducing | | | |

Agitation showed more severe stress condition compared to freeze/thaw stress. All samples looked hazy with visible particles and failed SVP specifications per USP<789> after 5-day agitation. Chemical stability after agitation was unchanged.

TABLE 5
shows characteristics after agitation.

| Test | IVT-1P | IVT-2 | IVT-4 |
|---|---|---|---|
| Concentration, | 201 | 180 | 198 |
| mg/mL | | | |
| Visible Particulates | Few Visible | Visible Particles | Visible Particles |
| | Particles | | |
| Clarity and | Less haziness | Slight haziness | Haziness |
| Opalescence | Btw. Std II-III | Eqv. Std III | Eqv. Std III |
| Sub Visible | 1194/18 | 160/11 | 993/35 |
| Particles, particles | | | |
| per mL, 10 μm/25 | | | |
| μm | | | |
| SE-HPLC | Main: 98.3% | Main: 98.5% | Main: 98.2% |
| | HMW: 1.2% | HMW: 1.4% | HMW: 1.7% |
| | LMW: 0.5% | LMW: 0.0% | LMW: 0.1% |
| CE-SDS, reducing | HC + LC: 100% | HC + LC: 100% | HC + LC: 100% |
| CE-SDS, non- | Intact: 100% | Intact: 100% | Intact: 100% |
| reducing | | | |

Accelerated stability was checked at 8 weeks at 25° C. All samples were within specifications except for SVP.

TABLE 6
shows stability data at 8 weeks at 25° C.

| Test | IVT-1P | IVT-2 | IVT-4 |
|---|---|---|---|
| Concentration, | 206 | 184 | 200 |
| mg/mL | | | |
| Visible Particulates | No Visible Particles | No Visible Particles | No Visible Particles |
| | observed | observed | observed |
| Clarity and | Eqv. Std II-III, | Btw. Std II-III, | Eqv. Std III |
| Opalescence | closer to III | closer to III | |
| pH | 6.62 | 6.60 | 6.77 |
| Sub Visible | 468/108 | 186/47 | 434/7 |
| Particles, particles | | | |
| per mL, 10 μm/25 | | | |
| μm | | | |
| SE-HPLC | Main: 96.9% | Main: 98.6% | Main: 98.6% |
| | HMW: 1.4% | HMW: 1.3% | HMW: 1.3% |
| | LMW: 1.7% | LMW: 0.0% | LMW: 0.0% |
| CE-SDS, reducing | HC + LC: 99% | HC + LC: 99% | HC + LC: 99% |
| CE-SDS, non- | Intact: 99% | Intact: 99% | Intact: 98% |
| reducing | | | |
| CEX-HPLC | Peak 1 + | Peak 1 + | Peak 1 + |
| | Peak 2: 90.3% | Peak 2: 91.4% | Peak 2: 89.5% |
| | Pre: 6.0% | Pre: 4.9% | Pre: 6.6% |
| | Inter: 2.9% | Inter: 3.0% | Inter: 3.1% |
| | Post: 0.6% | Post: 0.6% | Post: 0.7% |

Accelerated stability was checked at 8 weeks at 40° C. All samples were within specifications except for SVP. Other notable, albeit still acceptable, changes included increasing percentages of Pre-peak and Inter-peak species by cation exchange HPLC (CEX-HPLC) and high molecular weight species (HMW) by SEC-HPLC.

TABLE 7
shows stability data at 8 weeks at 40° C.

| Test | IVT-1P | IVT-2 | IVT-4 |
|---|---|---|---|
| Concentration, | 209 | 184 | 204 |
| mg/mL | | | |
| Visible Particulates | No Visible Particles | No Visible Particles | No Visible Particles |
| | observed | observed | observed |
| Clarity and | Eqv. Std II | Btw. Std II-III, | Eqv. Std II |
| Opalescence | | closer to III | |
| pH | 6.60 | 6.58 | 6.82 |
| Sub Visible | 388/33 | 185/3 | 553/16 |
| Particles, particles | | | |
| per mL, 10 μm/ | | | |
| 25 μm | | | |
| SE-HPLC | Main: 94.7% | Main: 97.1% | Main: 95.3% |
| | HMW: 3.5% | HMW: 2.8% | HMW: 4.7% |
| | LMW: 1.8% | LMW: 0.1% | LMW: 0.0% |
| CE-SDS, reducing | HC + LC: 98% | HC + LC: 98% | HC + LC: 97% |
| CE-SDS, non- | Intact: 95% | Intact: 96% | Intact: 94% |
| reducing | | | |
| CEX-HPLC | Peak 1 + | Peak 1 + | Peak 1 + |
| | Peak 2: 79.9% | Peak 2: 83.7% | Peak 2: 78.7% |
| | Pre: 11.0% | Pre: 8.5% | Pre: 12.0% |
| | Inter: 7.8% | Inter: 6.8% | Inter: 8.1% |
| | Post: 1.3% | Post: 0.9% | Post: 1.1% |

After freeze/thaw cycling and agitation, all samples failed visual appearance and SVP but passed all other product specifications. All samples passed product specifications, except SVP, after 8 weeks of storage at 25° C. and 40° C.

The surfactant level in the formulation was optimized and visible appearance acceptance criteria (i.e., no haziness, no visible particles) after at least 3 freeze/thaw cycles and after at least 4 weeks storage at 25° C.

Subsequent studies focused on SVP and visual appearance testing, which prove to be most stability indicating compared to other tests for measuring changes after multiple freeze-thaw cycling and agitation stress.

Optimal surfactant concentration was determined within a range of 0.02% to 0.06%. Further, Polysorbate 20 (PS20) and Polysorbate 80 (PS80) surfactants were compared at a fixed concentration of 0.06%. The study was performed at anti-C1q antibody Fab fragments concentration of 100 mg/mL.

TABLE 8
shows compositions of formulation candidates
at 100 mg/mL anti-C1q antibody Fab fragment.

| Formulation | Composition |
|---|---|
| IVT-2a | 10 mM sodium succinate, 5% (w/v) trehalose dihydrate, 40 mM |
| | NaCl, 0.02% (w/v) PS20, pH 6.4 |
| IVT-2b | 10 mM sodium succinate, 5% (w/v) trehalose dihydrate, 40 mM |
| | NaCl, 0.04% (w/v) PS20, pH 6.4 |
| IVT-2c | 10 mM sodium succinate, 5% (w/v) trehalose dihydrate, 40 mM |
| | NaCl, 0.06% (w/v) PS20, pH 6.4 |
| IVT-2d | 10 mM sodium succinate, 5% (w/v) trehalose dihydrate, 40 mM |
| | NaCl, 0.06% (w/v) PS80, pH 6.4 |

TABLE 9
shows characteristics of formulation candidates at t = 0.

| | | Visual Inspection for | Degree of Opalescence |
|---|---|---|---|
| Formulation | | Particulates | and Clarity |
| IVT-2a (0.02% | 3 freeze/thaw | No particulates | Opalescence not more |
| (w/v) PS20) | cycles | | pronounced than water |
| | 3 days agitation | No particulates | Opalescence not more |
| | | | pronounced than water |
| IVT-2b (0.04% | 3 freeze/thaw | No particulates | Opalescence not more |
| (w/v) PS20) | cycles | | pronounced than water |
| | 3 days agitation | No particulates | Opalescence not more |
| | | | pronounced than water |
| IVT-2c (0.06% | 3 freeze/thaw | No particulates | Opalescence not more |
| (w/v) PS20) | cycles | | pronounced than water |
| | 3 days agitation | No particulates | Opalescence not more |
| | | | pronounced than water |
| IVT-2d (0.06% | 3 freeze/thaw | Essentially free of | Opalescence not more |
| (w/v) PS80) | cycles | particulates | pronounced than water |
| | 3 days agitation | No particulates | Opalescence not more |
| | | | pronounced than water |

There was no significant difference between PS20 and PS80. Minimum surfactant (PS20) concentration of 0.04% (w/v) was required based on SVP results.

TABLE 10
shows characteristics after freeze/thaw cycling and agitation.

| | | | | | SVP, |
|---|---|---|---|---|---|
| | | | | | particles per |
| | | Conc, | Visual | Opalescence | mL (10 μm/ |
| Formulation | | mg/mL | Inspection | & Clarity | 25 μm) |
| IVT-2a | 3 freeze/thaw | 103.0 | No | Opalescence | 4223/83 |
| (0.02% (w/v) | cycles | | particulates | not more | |
| PS20) | | | | pronounced | |
| | | | | than water | |
| | 3 days | 104.0 | No | Opalescence | 10450/963 |
| | agitation | | particulates | not more | |
| | | | | pronounced | |
| | | | | than water | |
| IVT-2b | 3 freeze/thaw | 102.1 | No | Opalescence | 1743/10 |
| (0.04% (w/v) | cycles | | particulates | not more | |
| PS20) | | | | pronounced | |
| | | | | than water | |
| | 3 days | 102.7 | No | Opalescence | 2053/27 |
| | agitation | | particulates | not more | |
| | | | | pronounced | |
| | | | | than water | |
| IVT-2c | 3 freeze/thaw | 102.8 | No | Opalescence | 2270/77 |
| (0.06% (w/v) | cycles | | particulates | not more | |
| PS20) | | | | pronounced | |
| | | | | than water | |
| | 3 days | 103.2 | No | Opalescence | 2983/10 |
| | agitation | | particulates | not more | |
| | | | | pronounced | |
| | | | | than water | |
| IVT-2d | 3 freeze/thaw | 103.4 | Essentially | Opalescence | 2703/27 |
| (0.06% (w/v) | cycles | | free of | not more | |
| PS80) | | | particulates | pronounced | |
| | | | | than water | |
| | 3 days | 101.7 | No | Opalescence | 1437/13 |
| | agitation | | particulates | not more | |
| | | | | pronounced | |
| | | | | than water | |

Effect of anti-C1q antibody Fab fragment concentration was evaluated at 100 mg/mL and 200 mg/mL in 0.06% (w/v) PS20. Lowering the protein concentration did not result in being able to lower the surfactant concentration accordingly.

TABLE 11
shows effect of anti-C1q antibody Fab fragments concentrations.

| | | | | | SVP, |
|---|---|---|---|---|---|
| | | | | | particles |
| | | Conc, | Visual | Opalescence | per mL (10 |
| Formulation | | mg/mL | Inspection | & Clarity | μm/25 μm) |
| IVT-2c | 3 freeze/thaw | 102.8 | No | Opalescence | 2270/77 |
| (0.06% (w/v) | cycles | | particulates | not more | |
| PS20) | | | | pronounced | |
| | | | | than water | |
| | 3 days | 103.2 | No | Opalescence | 2983/10 |
| | agitation | | particulates | not more | |
| | | | | pronounced | |
| | | | | than water | |
| IVT-2c | 3 freeze/thaw | ~198.5 | No | Opalescence | 160/3 |
| (0.06% (w/v) | cycles | | particulates | not more | |
| PS20) | | | | pronounced | |
| | | | | than water | |
| | 3 days | ~198.5 | No | Opalescence | 573/20 |
| | agitation | | particulates | not more | |
| | | | | pronounced | |
| | | | | than water | |

The effect of pH was evaluated using sodium succinate and sodium citrate buffers at different pH.

TABLE 12
shows the composition of formulation candidates.

| Formulation | Composition |
|---|---|
| IVT-2_5.5 | 10 mM sodium succinate, 5% (w/v) trehalose dihydrate, 40 mM |
| | sodium chloride, 0.06% (w/v) PS20, pH 5.5 |
| IVT-2_6.4 | 10 mM sodium succinate, 5% (w/v) trehalose dihydrate, 40 mM |
| | sodium chloride, 0.06% (w/v) PS20, pH 6.4 |
| IVT-2_7.2 | 10 mM sodium succinate, 5% (w/v) trehalose dihydrate, 40 mM |
| | sodium chloride, 0.06% (w/v) PS20, pH 7.2 |
| IVT-5_6.4 | 10 mM sodium citrate, 5% (w/v) trehalose dihydrate, 40 mM sodium |
| | chloride, 0.06% (w/v) PS20, pH 6.4 |
| IVT-5_7.2 | 10 mM sodium citrate, 5% (w/v) trehalose dihydrate, 40 mM sodium |
| | chloride, 0.06% (w/v) PS20, pH 7.2 |

Sodium succinate pH 5.5 sample turned into a gel when stored at 2-8° C. However, the gel state was reversible and turned liquid at room temperature. Both sodium succinate and sodium citrate pH 6.4 samples went through this brief but noticeable gel phase while thawing, though remained liquid at 2-8° C. after complete thawing. However, all samples formulated at pH 7.2 did not exhibit any gelling behavior, transient or otherwise.

TABLE 13
shows characteristics after freeze/thaw cycling and agitation.

| | | Effect of pH, | | Effect of Succinate vs | |
|---|---|---|---|---|---|
| | | particles per mL* | | Citrate, particles per mL* | |
| Formulation | | 10 μm | 25 μm | 10 μm | 25 μm |
| IVT-2_5.5 | 3 freeze/thaw | 54.67 | 6.67 | NA | NA |
| | cycles | | | | |
| | 3 days agitation | 20.00 | 0.00 | NA | NA |
| IVT-2_6.4 | 3 freeze/thaw | 28.00 | 0.67 | NA | NA |
| | cycles | | | | |
| | 3 days agitation | 115.33 | 95.33 | NA | NA |
| IVT-2_7.2 | 3 freeze/thaw | 21.33 | 0.00 | 7 | 2 |
| | cycles | | | | |
| | 3 days agitation | 11.33 | 2.00 | 106 | 4 |
| IVT-5_6.4 | 3 freeze/thaw | NA | NA | 108 | 2 |
| | cycles | | | | |
| | 3 days agitation | NA | NA | 102 | 2 |
| IVT-5_7.2 | 3 freeze/thaw | NA | NA | 50 | 5 |
| | cycles | | | | |
| | 3 days agitation | NA | NA | 7 | 0 |

A pH-dependent gel phase transition was observed, showing a trend of product gelling with decreasing pH. Further, no difference in SVP was observed between sodium succinate and sodium citrate at pH 7.2.

Anti-C1q antibody Fab fragments were formulated in 10 mM sodium citrate, 50 (w/v) trehalose dihydrate, 40 mM sodium chloride, 0.06% (w/v) polysorbate 20, pH 7.2 and placed on stability.

TABLE 14
shows the stability at the selected formulation at −70° C.

| Test | Specification | t = 0 | t = 1 month |
|---|---|---|---|
| Degree of | Report result | Between BY5 and | Between BY5 and |
| Coloration | | BY6 | BY6 |
| Clarity and Degree | Less opalescent than | Between RSI and | Clear |
| of Opalescene | or equal to reference | RSII | |
| | suspension IV | | |
| Concentration | 200.0 ± 20.0 mg/mL | 202.0 | 203.2 |
| pH at 24 ± 1° C. | 7.2 ± 0.3 (pH units) | 7.1 | 7.0 |
| icIEF | Report comparison | CR | CR |
| | to reference standard | | |
| | Report pI range² | 8.25 to 8.77 | 8.15 to 8.88 |
| | Report pI of main | 8.68 | 8.75 |
| | isoform | | |
| | Report % main | 93.6 | 92.4 |
| | isoform | | |
| | Report % total acidic | 6.4 | 7.6 |
| | isoforms | | |
| | Report % total basic | ND | ND |
| | isoforms | | |
| GP HPLC¹ | Main peak: ≥95.0% | 99.3 | 99.3 |
| | Report % HMW | 0.7 | 0.7 |
| | Report % LMW | <0.1 | <0.1 |
| CE-SDS, reducing | HC + LC: ≥90.0 | 99.1 | 99.0 |
| | Report % LC | 44.3 | 43.4 |
| | Report % HC | 54.8 | 55.5 |
| CE-SDS, non- | Main peak: ≥80.0% | 98.2 | 98.2 |
| reducing | | | |
| Activity ELISA | 50% to 150% | 116 | 98 |
| | activity relative to | | |
| | reference standard | | |

BY = Brown (B)/Yellow (Y)
CR = Comparable to reference
Clear = Less than or equal to reference suspension I
ND = Not detected
HC = Heavy Chain
HMW = High Molecular Weight Species
LC = Light Chain
LMW = Low Molecular Weight Species
RS = Reference suspension
¹same method as SEC-HPLC
²pI range determined from pI start of first peak to pI end of last peak

TABLE 15
shows the stability at the selected formulation at 5° C.

| Test | Specification | t = 0 | t = 1 month |
|---|---|---|---|
| Degree of | Report result | Between BY5 and | Between BY5 and |
| Coloration | | BY6 | BY6 |
| Clarity and Degree | Less opalescent than | Between RSI and | Clear |
| of Opalescene | or equal to reference | RSII | |
| | suspension IV | | |
| Concentration | 200.0 ± 20.0 mg/mL | 202.0 | 200.6 |
| pH at 24 ± 1° C. | 7.2 ± 0.3 (pH units) | 7.1 | 7.1 |
| icIEF | Report comparison | CR | CR |
| | to reference standard | | |
| | Report pI range² | 8.25 to 8.77 | 8.16 to 8.86 |
| | Report pI of main | 8.68 | 8.75 |
| | isoform | | |
| | Report % main | 93.6 | 92.2 |
| | isoform | | |
| | Report % total acidic | 6.4 | 7.8 |
| | isoforms | | |
| | Report % total basic | ND | ND |
| | isoforms | | |
| GP HPLC¹ | Main peak: ≥95.0% | 99.3 | 99.2 |
| | Report % HMW | 0.7 | 0.7 |
| | Report % LMW | <0.1 | <0.1 |
| CE-SDS, reducing | HC + LC: ≥90.0 | 99.1 | 99.0 |
| | Report % LC | 44.3 | 43.6 |
| | Report % HC | 54.8 | 55.4 |
| CE-SDS, non- | Main peak: ≥80.0% | 98.2 | 98.2 |
| reducing | | | |
| Activity ELISA | 50% to 150% | 116 | 105 |
| | activity relative to | | |
| | reference standard | | |

BY = Brown (B)/Yellow (Y)
CR = Comparable to reference
Clear = Less than or equal to reference suspension I
ND = Not detected
HC = Heavy Chain
HMW = High Molecular Weight Species
LC = Light Chain
LMW = Low Molecular Weight Species
RS = Reference suspension
¹same method as SEC-HPLC
²pI range determined from pI start of first peak to pI end of last peak

TABLE 16
shows the stability at the selected formulation at 25° C.

| Test | Specification | t = 0 | t = 1 month |
|---|---|---|---|
| Degree of | Report result | Between BY5 and | Between BY5 and |
| Coloration | | BY6 | BY6 |
| Clarity and Degree | Less opalescent than | Between RSI and | Clear |
| of Opalescene | or equal to reference | RSII | |
| | suspension IV | | |
| Concentration | 200.0 ± 20.0 mg/mL | 202.0 | 201.5 |
| pH at 24 ± 1° C. | 7.2 ± 0.3 (pH units) | 7.1 | 7.1 |
| icIEF | Report comparison | CR | CR |
| | to reference standard | | |
| | Report pI range² | 8.25 to 8.77 | 8.15 to 8.89 |
| | Report pI of main | 8.68 | 8.76 |
| | isoform | | |
| | Report % main | 93.6 | 92.1 |
| | isoform | | |
| | Report % total acidic | 6.4 | 7.9 |
| | isoforms | | |
| | Report % total basic | ND | ND |
| | isoforms | | |
| GP HPLC¹ | Main peak: ≥95.0% | 99.3 | 99.0 |
| | Report % HMW | 0.7 | 1.0 |
| | Report % LMW | <0.1 | <0.1 |
| CE-SDS, reducing | HC + LC: ≥90.0 | 99.1 | 98.8 |
| | Report % LC | 44.3 | 43.7 |
| | Report % HC | 54.8 | 55.2 |
| CE-SDS, non- | Main peak: ≥80.0% | 98.2 | 97.8 |
| reducing | | | |
| Activity ELISA | 50% to 150% | 116 | 94 |
| | activity relative to | | |
| | reference standard | | |

BY = Brown (B)/Yellow (Y)
CR = Comparable to reference
Clear = Less than or equal to reference suspension I
ND = Not detected
HC = Heavy Chain
HMW = High Molecular Weight Species
LC = Light Chain
LMW = Low Molecular Weight Species
RS = Reference suspension
¹same method as SEC-HPLC
²pI range determined from pI start of first peak to pI end of last peak

### INCORPORATION BY REFERENCE

Each of the patents, published patent applications, and non-patent references cited herein are hereby incorporated by reference in their entirety.

### EQUIVALENTS

Those skilled in the art will recognize or be able to ascertain using no more than routine experimentation, many equivalents to the specific embodiments of the invention described herein. Such equivalents are intended to be encompassed by the following claims.

1. A pharmaceutical composition comprising:
(a) at least 75 mg/ml of an anti-C1q antibody or an antigen-binding fragment thereof;
(b) from 5 to 25 mM sodium citrate or sodium succinate;
(c) from 3% to 7% trehalose; and
(d) from 0.04% to 0.06% polysorbate 20 or polysorbate 80,
wherein the pharmaceutical composition is at a pH of 6.4-7.5.

2. The pharmaceutical composition of claim 1, wherein the antibody or an antigen-binding fragment thereof comprises a light chain variable domain comprising an HVR-L1 having the amino acid sequence of SEQ ID NO: 5, an HVR-L2 having the amino acid sequence of SEQ ID NO: 6, and an HVR-L3 having the amino acid sequence of SEQ ID NO: 7 and a heavy chain variable domain comprising an HVR-H1 having the amino acid sequence of SEQ ID NO: 9, an HVR-H2 having the amino acid sequence of SEQ ID NO: 10, and an HVR-H3 having the amino acid sequence of SEQ ID NO: 11.

3. (canceled)

4. The pharmaceutical composition of claim 1, wherein the antibody or an antigen-binding fragment thereof is a monoclonal antibody, a polyclonal antibody, a recombinant antibody, a humanized antibody, a chimeric antibody, a multispecific antibody, or an antibody derivative thereof.

5. (canceled)

6. The pharmaceutical composition of claim 1, wherein the antibody or an antigen-binding fragment thereof comprises a light chain variable domain comprising an amino acid sequence with at least about 95% homology to the amino acid sequence selected from any one of SEQ ID NO: 4 and 35-38 and wherein the light chain variable domain comprises an HVR-L1 having the amino acid sequence of SEQ ID NO: 5, an HVR-L2 having the amino acid sequence of SEQ ID NO: 6, and an HVR-L3 having the amino acid sequence of SEQ ID NO: 7 and a heavy chain variable domain comprising an amino acid sequence with at least about 95% homology to the amino acid sequence selected from any one of SEQ ID NO: 8 and 31-34 and wherein the heavy chain variable domain comprises an HVR-H1 having the amino acid sequence of SEQ ID NO: 9, an HVR-H2 having the amino acid sequence of SEQ ID NO: 10, and an HVR-H3 having the amino acid sequence of SEQ ID NO: 11.

7. The pharmaceutical composition of claim 6, wherein the light chain variable domain comprises an amino acid sequence selected from any one of SEQ ID NO: 4 and 35-38 and the heavy chain variable domain comprises an amino acid sequence selected from any one of SEQ ID NO: 8 and 31-34.

8-9. (canceled)

10. The pharmaceutical composition of claim 1, wherein the antibody is an antigen-binding fragment.

11. The pharmaceutical composition of claim 10, wherein the antigen-binding fragment is a Fab fragment, a Fab′ fragment, a F(ab′)2 fragment, a Fv fragment, a diabody, or a single chain antibody molecule.

12. The pharmaceutical composition of claim 11, wherein the antibody Fab fragment comprises a heavy chain Fab fragment of SEQ ID NO: 39 and a light chain Fab fragment of SEQ ID NO: 40.

13. (canceled)

14. The pharmaceutical composition of claim 1, wherein the pharmaceutical composition comprises at least 100 mg/ml, at least 125 mg/ml, at least 150 mg/ml, at least 175 mg/ml, at least 200 mg/ml, at least 225 mg/ml, or at least 250 mg/ml of the anti-C1q antibody or antigen-binding fragment thereof.

15-16. (canceled)

17. The pharmaceutical composition of claim 1, wherein the pharmaceutical composition comprises about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, or about 25 mM sodium citrate or sodium succinate.

18. (canceled)

19. The pharmaceutical composition of claim 1, wherein the pharmaceutical composition comprises about 3%, about 4%, about 5%, about 6%, or about 7% trehalose.

20. The pharmaceutical composition of claim 1, wherein the pharmaceutical composition comprises about 0.04%, about 0.05%, or about 0.06% polysorbate 20 or polysorbate 80.

21. The pharmaceutical composition of claim 1, wherein the pharmaceutical composition is at a pH of about 6.4, about 6.5, about 6.6, about 6.7, about 6.8, about 6.9, about 7.0, about 7.1, about 7.2, about 7.3, about 7.4, or about 7.5.

22. The pharmaceutical composition of claim 1, wherein the pharmaceutical composition further comprises from 30 mM to 50 mM NaCl.

23. (canceled)

24. The pharmaceutical composition of claim 1, wherein the viscosity of the pharmaceutical composition is no more than 10 cP at 25° C.

25. (canceled)

26. The pharmaceutical composition of claim 1, wherein the pharmaceutical composition is stable for at least 1 week, at least 2 weeks, at least 3 weeks, at least 4 weeks, at least 5 weeks, at least 6 weeks, at least 7 weeks, at least 8 weeks, at least 9 weeks, at least 10 weeks, at least 11 weeks, at least 12 weeks, at least 13 weeks, at least 14 weeks, at least 15 weeks, at least 16 weeks at least 17 weeks, at least 18 weeks, at least 19 weeks, at least 20 weeks, at least 21 weeks, at least 22 weeks, at least 23 weeks, or at least 24 weeks.

27-49. (canceled)

50. An injector comprising the pharmaceutical composition of claim 1.

51-56. (canceled)

57. A prefilled syringe comprising the pharmaceutical composition of claim 1.

58-61. (canceled)

62. A method of inhibiting synapse loss, comprising administering to a patient suffering from adverse synapse loss the pharmaceutical composition claim 1.

63-65. (canceled)

66. A method of treating or preventing a disease associated with complement activation in an individual in need of such treatment, the method comprising administering the pharmaceutical composition of claim 1.

67-78. (canceled)


SEQUENCE LISTING (51 sequences)

SEQ ID NO: 1 - protein, 245 aa, synthetic construct
MEGPRGWLVLCVLAISLASMVTEDLCRAPDGKKGEAGRPGRRGRPGLKGEQGEPGAPGIRTGIQGLKGDQGEPGPSGNPGKVGYPGPSGPLGARGIPGIKGTKGSPGNIKDQPRPAFSAIRRNPPMGGNVVIFDTVITNQEEPYQNHSGRFVCTVPGYYYFTFQVLSQWEICLSIVSSSRGQVRRSLGFCDTTNKGLFQVVSGGMVLQLQQGDQVWVEKDPKKGHIYQGSEADSVFSGFLIFPSA

SEQ ID NO: 2 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 4 - protein, 107 aa, synthetic construct
DVQITQSPSYLAASPGETITINCRASKSINKYLAWYQEKPGKTNKLLIYSGSTLQSGIPSRFSGSGSGTDFTLTISSLEPEDFAMYYCQQHNEYPLTFGAGTKLELK

SEQ ID NO: 5 - protein, 11 aa, synthetic construct
RASKSINKYLA

SEQ ID NO: 6 - protein, 7 aa, synthetic construct
SGSTLQS

SEQ ID NO: 7 - protein, 9 aa, synthetic construct
QQHNEYPLT

SEQ ID NO: 8 - protein, 121 aa, synthetic construct
QVQLQQPGAELVKPGASVKLSCKSSGYHFTSYWMHWVKQRPGQGLEWIGVIHPNSGSINYNEKFESKATLTVDKSSSTAYMQLSSLTSEDSAVYYCAGERDSTEVLPMDYWGQGTSVTVSS

SEQ ID NO: 9 - protein, 10 aa, synthetic construct
GYHFTSYWMH

SEQ ID NO: 10 - protein, 17 aa, synthetic construct
VIHPNSGSINYNEKFES

SEQ ID NO: 11 - protein, 12 aa, synthetic construct
ERDSTEVLPMDY

SEQ ID NO: 12 - protein, 321 aa, synthetic construct
GATGTCCAGATAACCCAGTCTCCATCTTATCTTGCTGCATCTCCTGGAGAAACCATTACTATTAATTGCAGGGCAAGTAAGAGCATTAACAAATATTTAGCCTGGTATCAAGAGAAACCTGGGAAAACTAATAAGCTTCTTATCTACTCTGGATCCACTTTGCAATCTGGAATTCCATCAAGGTTCAGTGGCAGTGGATCTGGTACAGATTTCACTCTCACCATCAGTAGCCTGGAGCCTGAAGATTTTGCAATGTATTACTGTCAACAACATAATGAATACCCGCTCACGTTCGGTGCTGGGACCAAGCTGGAGCTGAAA

SEQ ID NO: 13 - protein, 363 aa, synthetic construct
CAGGTCCAACTGCAGCAGCCTGGGGCTGAGCTGGTAAAGCCTGGGGCTTCAGTGAAGTTGTCCTGCAAGTCTTCTGGCTACCATTTCACCAGCTACTGGATGCACTGGGTGAAGCAGAGGCCTGGACAAGGCCTTGAGTGGATTGGAGTGATTCATCCTAATAGTGGTAGTATTAACTACAATGAGAAGTTCGAGAGCAAGGCCACACTGACTGTAGACAAATCCTCCAGCACAGCCTACATGCAACTCAGCAGCCTGACATCTGAGGACTCGGCGGTCTATTATTGTGCAGGAGAGAGAGATTCTACGGAGGTTCTCCCTATGGACTACTGGGGTCAAGGAACCTCAGTCACCGTCTCCTCA

SEQ ID NO: 14 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 121 aa, synthetic construct; identical to SEQ ID NO: 8

SEQ ID NO: 16 - protein, 121 aa, synthetic construct [sequence omitted]

SEQ ID NO: 17 - protein, 121 aa, synthetic construct [sequence omitted]

SEQ ID NO: 18 - protein, 121 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSS

SEQ ID NO: 19 - protein, 121 aa, synthetic construct [sequence omitted]

SEQ ID NO: 20 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 451 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - skipped

SEQ ID NO: 23 - protein, 253 aa, synthetic construct
MMMKIPWGSIPVLMLLLLLGLIDISQAQLSCTGPPAIPGIPGIPGTPGPDGQPGTPGIKGEKGLPGLAGDHGEFGEKGDPGIPGNPGKVGPKGPMGPKGGPGAPGAPGPKGESGDYKATQKIAFSATRTINVPLRRDQTIRFDHVITNMNNNYEPRSGKFTCKVPGLYYFTYHASSRGNLCVNLMRGRERAQKVVTFCDYAYNTFQVTTGGMVLKLEQGENVFLQATDKNSLLGMEGANSIFSGFLLFPDMEA

SEQ ID NO: 24 - protein, 245 aa, synthetic construct
MDVGPSSLPHLGLKLLLLLLLLPLRGQANTGCYGIPGMPGLPGAPGKDGYDGLPGPKGEPGIPAIPGIRGPKGQKGEPGLPGHPGKNGPMGPPGMPGVPGPMGIPGEPGEEGRYKQKFQSVFTVTRQTHQPPAPNSLIRFNAVLTNPQGDYDTSTGKFTCKVPGLYYFVYHASHTANLCVLLYRSGVKVVTFCGHTSKTNQVNSGGVLLRLQVGEEVWLAVNDYYDMVGIQGSDSVFSGFLLFPD

SEQ ID NO: 25 - protein, 330 aa, synthetic construct [sequence omitted]

SEQ ID NO: 26 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27-30 - skipped (4 entries)

SEQ ID NO: 31 - protein, 121 aa, synthetic construct [sequence omitted]

SEQ ID NO: 32 - protein, 121 aa, synthetic construct [sequence omitted]

SEQ ID NO: 33 - protein, 121 aa, synthetic construct; identical to SEQ ID NO: 18

SEQ ID NO: 34 - protein, 121 aa, synthetic construct [sequence omitted]

SEQ ID NO: 35 - protein, 107 aa, synthetic construct [sequence omitted]

SEQ ID NO: 36 - protein, 107 aa, synthetic construct [sequence omitted]

SEQ ID NO: 37 - protein, 107 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIK

SEQ ID NO: 38 - protein, 107 aa, synthetic construct [sequence omitted]

SEQ ID NO: 39 - protein, 229 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGERDSTEVLPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 40 - protein, 214 aa, synthetic construct
DVQITQSPSSLSASLGERATINCRASKSINKYLAWYQQKPGKAPKLLIYSGSTLQSGIPARFSGSGSGTDFTLTISSLEPEDFAMYYCQQHNEYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 43 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 44 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 45 - protein, 448 aa, synthetic construct
QVQLVQSGAELKKPGASVKVSCKSSGYHFTSYWMKWVKQAPGQGLEWIGVIHPNSGSINYNKKFESRVTITVDKSTSTAYMELSSLRSEDTAVYYCAGARKSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 46 - protein, 448 aa, synthetic construct
QVQLVQSGAELKKPGASVKVSCKSSGYHFTSYWMHWVKQAPGQGLEWIGVIHPNSGSINYNKKFKSRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 47 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGARASTAVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 48 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 49 - protein, 448 aa, synthetic construct
QVQLVQSGAELKKPGASVKVSCKSSGYHFTSYWMKWVKQAPGQGLEWIGVIHPNSGSINYNKKFESRVTITVDKSTSTAYMELSSLRSEDTAVYYCAGARKSTEVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 50 - protein, 448 aa, synthetic construct
QVQLVQSGAELKKPGASVKVSCKSSGYHFTSYWMHWVKQAPGQGLEWIGVIHPNSGSINYNKKFKSRVTITVDKSTSTAYMELSSLRSEDTAVYYCAGKRKSTKVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 51 - protein, 448 aa, synthetic construct
QVQLVQSGAELKKPGASVKVSCKSSGYHFTSYWMHWVKQAPGQGLEWIGVIHPNSGSINYNEKFESRVTITVDKSTSTAYMELSSLRSEDTAVYYCAGARASTAVLPMDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK